(19) United States
(12) Patent Application Publication
(10) Pub. No.: US 2013/0310611 A1
(43) Pub. Date: Nov. 21, 2013

(54) PROCESS FOR PRODUCTION OF PHENOLIC POLYMERIZABLE COMPOUND HAVING PHYSIOLOGICAL ACTIVITY

(71) Applicants: Akinobu Kishi, Yamatokooriyama-shi (JP); Satoshi Doi, Yamatokooriyama-shi (JP); Taiji Matsukawa, Yamatokooriyama-shi (JP); Takeki Matsui, Yamatokooriyama-shi (JP); Yasumasa Yamada, Yamatokooriyama-shi (JP); Ichiro Yamada, Yamatokooriyama-shi (JP)

(72) Inventors: Akinobu Kishi, Yamatokooriyama-shi (JP); Satoshi Doi, Yamatokooriyama-shi (JP); Taiji Matsukawa, Yamatokooriyama-shi (JP); Takeki Matsui, Yamatokooriyama-shi (JP); Yasumasa Yamada, Yamatokooriyama-shi (JP); Ichiro Yamada, Yamatokooriyama-shi (JP)

(73) Assignee: UHA MIKAKUTO CO., LTD., Yamatokooriyama-Shi, Naga (JP)

(21) Appl. No.: 13/882,032

(22) PCT Filed: Oct. 27, 2011
(86) PCT No.: PCT/JP2011/074869

§ 371 Date: Jul. 17, 2013

(30) Foreign Application Priority Data
Oct. 28, 2010 (JP) 2010-242661
Oct. 28, 2010 (JP) 2010-242673
Nov. 26, 2010 (JP) 2010-264173
Nov. 29, 2010 (JP) 2010-264779
Apr. 22, 2011 (JP) 2011-096213
Apr. 22, 2011 (JP) 2011-096223

(51) Int. Cl. C07C 37/11; C07C 39/21; C07C 43/23; C07C 41/01

(52) U.S. Cl. CPC C07C 37/11; C07C 41/01; C07C 39/21; C07C 43/23
USPC 568/646; 568/729; 568/720

(57) ABSTRACT

A process for producing a phenolic polymerizable compound represented by formula (1) or (2); wherein X¹-X⁹ independently represent a hydrogen atom, a hydroxy group, a saturated or unsaturated linear or branched alkoxy group having 1-10 carbon atoms, or a saturated or unsaturated linear or branched alkyl group having 1-10 carbon atoms; Y represents a hydrogen atom, a hydroxy group, a saturated or unsaturated linear or branched alkoxy group having 1-10 carbon atoms, a saturated or unsaturated linear or branched alkyl group having 1-10 carbon atoms, or a group represented by formula (6); and Z represents a hydrogen atom or a group represented by formula (3), which is characterized by heating a 4-hydroxycinnamic acid compound in the presence of a metal salt.

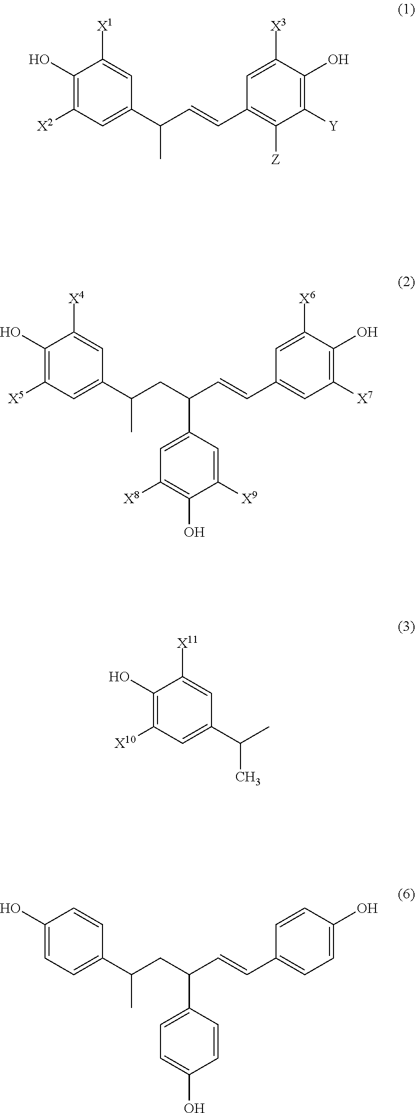

### TECHNICAL FIELD

The present invention relates to a process for production of a phenolic polymerizable compound having physiological activity containing 4-hydroxycinnamic acid compounds as raw materials. The invention also relates to anticancer agents, anticancer agents to oral cancer, lipase inhibitors, anti-obesity agents, skin disease therapeutic agents, foods, pharmaceutical agents, quasi-drugs, and cosmetics containing the physiologically active phenolic polymerizable compound. The invention also relates to a novel phenolic polymerizable compound.

### BACKGROUND ART

The 4-hydroxycinnamic acid compounds are secondary metabolites biosynthesized in plants mainly in the shikimic acid pathway and are compounds serving as raw materials of substances whose physiological activity have been reported in a large number of reports, such as phenyl propanoid, flavonoid, lignan, and tannin. The 4-hydroxycinnamic acid compounds themselves are also compounds which are biosynthesized from plants in order to protect the plants themselves and seeds from ultraviolet rays and a large amount of which are present in the nature. It is known that the phenolic polymerizable compound is contained in roasted foods, such as coffee, and contributes to bitter taste, for example. The content thereof is very small. With respect to flavonoid, lignan, and the like which are polymers of the 4-hydroxycinnamic acid compound, a large number of compounds having physiological activity are present and foods containing the same are eaten for health enhancement.

With respect to the generation reaction from the 4-hydroxycinnamic acid compounds to the phenolic polymerizable compounds, a chemical synthesis method of phenolic polymerizable compounds from ferulic acid, coffeic acid, and p-coumaric acid (Non-patent Document 1) is known. In the method, the phenolic polymerizable compound is obtained by increasing the temperature of the 4-hydroxycinnamic acid compound to a temperature of equal to or higher than the melting point of each compound, and holding the same at 200° C. or higher. Thus, it is clear that the target reaction does not proceed under the temperature conditions equal to or lower than the melting point, i.e., 150° C. or lower. In the method, the control of the manufacturing conditions, such as sealing and degassing, is complicated also in the process. Therefore, this method has serious problems in workability, safety, and the like for mass production. In a synthesis method described in Non-patent Document 2, a slight amount of the phenolic polymerizable compound is merely generated as a by-product. The synthesis methods described above each include a reaction from one kind of cinammic acid. A generation method from two or more kinds of cinammic acids is not described at all and the physiological activity of the phenolic polymerizable compound to be obtained is not described at all.

With respect to flavonoid, lignan, known phenolic polymerizable compounds, and the like, various physiological activities have been reported. However, in order to purify these compounds from natural products, there are a large number of problems in operation, such as increase in the cost and deterioration in the working efficiency. Further, when purified, a trace constituent is formed. Therefore, a method for easily obtaining the same has been desired.

### CITATION LIST

### Non-Patent Literatures

Non-patent Document 1: J. Agricultural and Food Chemistry, 1992, 40, 1666-1670
Non-patent Document 2: Polymer Journal. 1981, 13, 563-568

### SUMMARY OF INVENTION

### Technical Problem

In view of the above-described circumstances, the present inventors have conducted extensive researches in order to establish a process for production of a phenolic polymerizable compound. As a result, the present inventors have successively produced a dimer or trimer phenolic polymerizable compound excellent in physiological activities, such as anti-cancer activity, anti-cancer activity to oral cancer, and lipase inhibition activity, by a simple and safe process including heating 4-hydroxycinnamic acid compounds as the raw materials in the presence of a metal salt, and thus have accomplished the invention.

Therefore, it is an object of the invention to provide a process capable of efficiently and safely obtaining a dimer or trimer phenolic polymerizable compound having one or more kinds of physiological activities of anti-cancer activity, anti-cancer activity to oral cancer, and lipase inhibition activity.

It is another object of the present invention to provide anticancer agents, anticancer agents to oral cancer, lipase inhibitors, anti-obesity agents, skin disease therapeutic agents, foods, pharmaceutical agents, quasi-drugs, and cosmetics containing the phenolic polymerizable compound.

It is still another object of the present invention to provide a novel phenolic polymerizable compound having one or more kinds of physiological activities of anti-cancer activity, anti-cancer activity to oral cancer, and lipase inhibition activity.

### Solution to Problem

The gist of the invention relates to the following items:

[1] A process for producing a phenolic polymerizable compound represented by Formula (1):

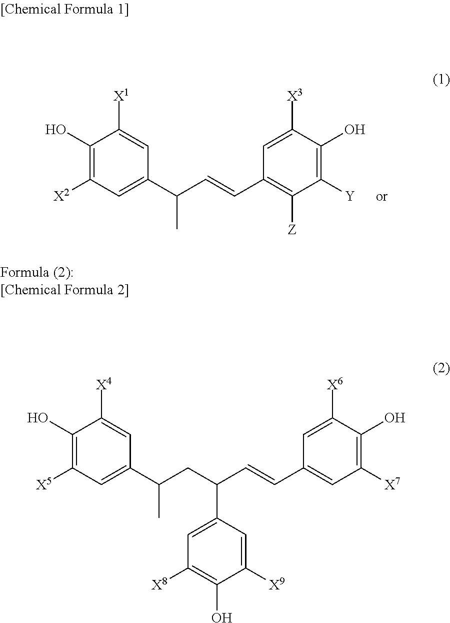

(in Formulae (1) and (2), X¹-X⁹ independently represent a hydrogen atom, a hydroxy group, a saturated or unsaturated linear or branched alkoxy group having 1-10 carbon atoms, or a saturated or unsaturated linear or branched alkyl group having 1-10 carbon atoms; Y represents a hydrogen atom, a hydroxy group, a saturated or unsaturated linear or branched alkoxy group having 1-10 carbon atoms, a saturated or unsaturated linear or branched alkyl group having 1-10 carbon atoms, or a group represented by Formula (3):

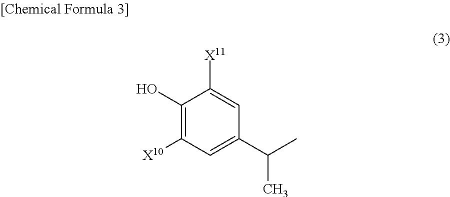

(in Formula (3), X¹⁰ and X¹¹ independently represent a hydrogen atom, a hydroxy group, a saturated or unsaturated linear or branched alkoxy group having 1-10 carbon atoms, or a saturated or unsaturated linear or branched alkyl group having 1-10 carbon atoms); and Z represents a hydrogen atom or a group represented by Formula (3); in which X¹-X¹¹ may be the same or different from one another), the process being characterized by heating a 4-hydroxycinnamic acid compound in the presence of a metal salt,

[2] An anticancer agent containing a phenolic polymerizable compound produced by the process described above,

[3] An anticancer agent to oral cancer containing a phenolic polymerizable compound produced by the process described above,

[4] A lipase inhibitor containing a phenolic polymerizable compound produced by the process described above,

[5] An anti-obesity agent containing a phenolic polymerizable compound produced by the process described above,

[6] A skin disease therapeutic agent containing a phenolic polymerizable compound produced by the process described above,

[7] A food, a pharmaceutical agent, a quasi-drug, or cosmetics containing a phenolic polymerizable compound produced by the process described above, and

[8] A novel physiologically active phenolic polymerizable compound or a pharmacologically permissible salt thereof represented by Formula (7):

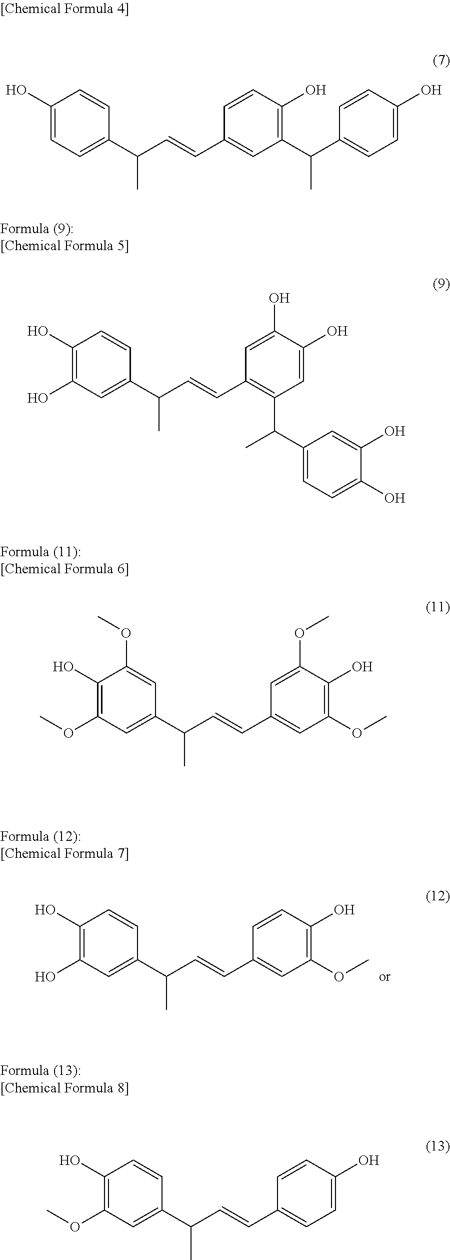

### Advantageous Effects of Invention

According to the present invention, a dimer or trimer phenolic polymerizable compound excellent in physiological activities, such as anti-cancer activity, anti-cancer activity to oral cancer, and lipase inhibition activity, can be efficiently and safely obtained.

The phenolic polymerizable compound obtained by the invention also serves as an active ingredient of anticancer agents, anticancer agents to oral cancer, lipase inhibitors, anti-obesity agents, and skin disease therapeutic agents and also, by compounding the phenolic polymerizable compound in foods, pharmaceutical agents, quasi-drugs, or cosmetics, the physiological activities can be newly imparted to these products or the physiological activities which are already imparted thereto can be further strengthened.

### BRIEF DESCRIPTION OF DRAWINGS

FIG. 1 show analysis results of high performance liquid chromatography (HPLC) performed in Example 1. The upper view shows the results before a reaction and the lower view shows the results after a reaction, in which “A”, “B”, and “C” represent peaks of phenolic polymerizable compounds generated using p-coumaric acid as raw materials.

FIG. 2 show the analysis results of HPLC performed in Example 5. The upper view shows the results before a reaction and the lower view shows the results after a reaction, in which “D” and “E” represent the peaks of phenolic polymerizable compounds generated using coffeic acid as the raw materials.

FIG. 3 show the analysis results of HPLC performed in Example 7. The upper view shows the results before a reaction and the lower view shows the results after a reaction, in which “F” represents the peak of a phenolic polymerizable compound generated using ferulic acid as the raw materials.

FIG. 4 show the analysis results of HPLC performed in Example 9. The upper view shows the results before a reaction and the lower view shows the results after a reaction, in which “G” represents the peak of a phenolic polymerizable compound generated using sinapic acid as the raw materials.

FIG. 5 show the analysis results of HPLC performed in Example 11. The upper view shows the results after a reaction in the case of only coffeic acid, the middle view shows the results after a reaction in the case of only ferulic acid, and the lower view shows the results after a reaction of a mixed liquid of coffeic acid and ferulic acid, in which “H” represents the peak of a phenolic polymerizable compound generated using coffeic acid and ferulic acid as the raw materials.

FIG. 6 show the analysis results of HPLC performed in Example 13. The upper view shows the results after a reaction in the case of only p-coumaric acid, the middle view shows the results after a reaction in the case of ferulic acid, and the lower view shows the results after a reaction of a mixed liquid of p-coumaric acid and ferulic acid, in which “I” represents the peak of a phenolic polymerizable compound generated using p-coumaric acid and ferulic acid as the raw materials.

### DESCRIPTION OF EMBODIMENTS

Hereinafter, the invention is described in detail.

The invention is a process for production of a dimer or trimer phenolic polymerizable compound having physiological activity (hereinafter referred to as phenolic polymerizable compound) including a process for heating one or more kinds of 4-hydroxycinnamic acid compounds in the presence of a metal salt.

In the invention, the physiological activities refer to anti-cancer activity, anti-cancer activity to oral cancer, and lipase inhibition activity. A compound having one or more of the physiological activities is referred to as a “compound having physiological activity”. The dimer or trimer refers to one having two or three benzene portions derived from 4-hydroxycinnamic acid compounds.

The present inventors have conducted extensive research, and as a result have found that a dimer or trimer phenolic polymerizable compound having physiological activity can be efficiently and safely produced by heating 4-hydroxycinnamic acid compounds in the presence of a metal salt.

In the production process of the invention, 4-hydroxycinnamic acid compounds are used as the raw materials. The 4-hydroxycinnamic acid compounds may be a cinnamic acid compound having a hydroxyl group at the 4th position of the benzene portion and derivatives thereof. Specifically, compounds represented by the following formula (4) are given.

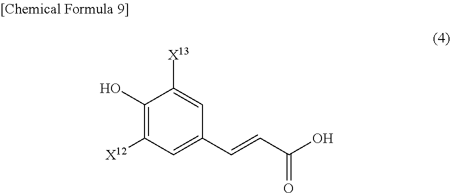

(in Formula (4), X¹² and X¹³ independently represent a hydrogen atom, a hydroxy group, a saturated or unsaturated linear or branched alkoxy group having 1-10 carbon atoms, or a saturated or unsaturated linear or branched alkyl group having 1-10 carbon atoms, and X¹² and X¹³ may be the same or different from each another.)

As the 4-hydroxycinnamic acid compounds, a 4-hydroxycinnamic acid compound having hydrogen at the 2nd position and the 6th position of the benzene portion and having the same functional group or different functional groups selected from hydrogen, a hydroxy group, a saturated or unsaturated linear or branched alkoxy group having 1-10 carbon atoms, and a saturated or unsaturated linear or branched alkyl group having 1-10 carbon atoms at the 3rd position and the 5th position of the benzene portion is preferable from the viewpoint of good generation efficiency during a reaction. From the view point of availability and cost, p-coumaric acid, ferulic acid, coffeic acid, sinapic acid, di-t-butyl hydroxycinnamic acid, artepillin C, and the like are more preferable.

In the production process of the invention, a phenolic polymerizable compound having a desired structure can be obtained by selecting the kind of the 4-hydroxycinnamic acid compounds as described later.

In the stages of the production process of the invention, it is considered that the 4-hydroxycinnamic acid compounds serving as the raw materials undergo decarboxylation, so that 4-vinyl phenols are generated, and then the reaction further proceeds so that phenolic polymerizable compounds which are dimers or trimers of the 4-vinyl phenols are generated.

The 4-hydroxycinnamic acid compounds as the raw materials may be those derived from nature or chemical products with high purity which are obtained by chemical synthesis. The naturally derived 4-hydroxycinnamic acid compounds are not required to be completely purified and a mixture containing the ingredients of the 4-hydroxycinnamic acid compounds can be also used. The 4-hydroxycinnamic acid compounds include derivatives, such as salts and esters. In the production process of the invention, these derivatives can be also used as the raw materials.

As the derivatives of the 4-hydroxycinnamic acid compounds, salts, such as sodium salt, potassium salt, and calcium salt, and esters, such as methyl ester and ethyl ester, are given.

From the viewpoint of increasing the generation efficiency and the recovery rate of the phenolic polymerizable compound, a mixture containing the 4-hydroxycinnamic acid compounds in a proportion of 10% by weight or more in total is preferable as the raw materials.

In the production process of the invention, the 4-hydroxycinnamic acid compounds are dissolved in a suitable solvent. In this case, when the solvent contains only water, the 4-hydroxycinnamic acid compounds may be dissolved in a mixed liquid of water and an organic solvent or only an organic solvent because the solubility of the 4-hydroxycinnamic acid compounds in water is remarkably low. In this case, the compounding ratio of water and the organic solvent and the kind of the organic solvent are not particularly limited insofar as the 4-hydroxycinnamic acid compounds are sufficiently dissolved. In particular, the use of a solvent containing only methanol or only ethanol or a mixed liquid of water and methanol or a mixed liquid of water and ethanol is excellent in safety and is preferable in terms of low cost. When using a composition (reactant) containing the phenolic polymerizable compound after the reaction for foods without sufficiently purifying the same, it is preferable to use ethanol or hydrous ethanol as the solvent in terms of safety, and laws and regulations.

The concentration of the 4-hydroxycinnamic acid compounds in the solution containing the 4-hydroxycinnamic acid compounds obtained as described above is not particularly limited. For example, when the concentration of the 4-hydroxycinnamic acid compounds is higher, there are merits such that the use amount of the solvent is small. Therefore, it is preferable to adjust the concentration of the 4-hydroxycinnamic acid compounds in such a manner as to be close to the concentration at which the compounds are saturated in each solvent.

In the production process of the invention, in order to react the 4-hydroxycinnamic acid compounds to obtain the target phenolic polymerizable compound, a metal salt is added to a solution containing the 4-hydroxycinnamic acid compounds (hereinafter referred to as a raw material solution).

In the production process of the invention, the reaction of generating the phenolic polymerizable compound proceeds as described later even under acidic conditions or alkaline conditions insofar as the reaction proceeds in the presence of a metal salt.

However, when the raw materials are compounds having two or more hydroxyl groups adjacent to each other in the benzene portion, such as coffeic acid or trihydroxycinnamic acid, a reaction other than the target generation reaction proceeds under alkaline conditions, and the recovery rate of the phenolic polymerizable compound decreases.

Accordingly, when the hydroxycinnamic acid compounds having two or more hydroxyl groups adjacent to each other in the benzene portion, e.g., coffeic acid and trihydroxycinnamic acid, are contained as the raw materials, the pH of the raw material solution when starting the reaction is desirably lower than 7.

When the hydroxycinnamic acid compound having two or more hydroxyl groups in the benzene portion are not contained as the raw materials, the conditions may be acidic conditions or alkaline conditions. However, considering only from the reaction efficiency, alkaline conditions are preferable. However, it is desirable to appropriately select the reaction conditions considering the intended use of the phenolic polymerizable compound to be obtained and the presence or absence of purifying operation and isolation operation and, when added to foods, considering the taste and the like.

In the production process of the invention, the generation reaction is performed in the presence of a metal salt. Specifically, a metal salt is added into the raw material solution. The metal salt may be any one of acidic salts, basic salts, and normal salts and may be any one of single salts, double salts, and complex salts. The metal salt may be one kind thereof or a mixture of two or more kinds thereof. As an example of the metal salt, one approved by the country and the international organization as food additives is preferable in terms of safety. For example, magnesium salt, calcium salt, sodium salt, potassium salt, zinc salt, copper salt, and the like which are approved to be added to foods are given.

The mixture of the metal salts includes, for example, a mixture containing several kinds of metal salts, such as Mineral premix (Tanabe Seiyaku Co., Ltd., mineral mixture containing zinc gluconate, iron ammonium citrate, calcium lactate, copper gluconate, and magnesium phosphate as the main ingredients). Moreover, mineral water can also be mentioned as the mixture containing a plurality of kinds of metal salts.

The content of the metal salt in the raw material solution is not particularly limited insofar as the phenolic polymerizable compound can be generated.

In the production process of the invention, the raw material solution may be heated as it is without adjusting the pH. In this case, although the raw material solution becomes acidic or alkaline depending on the amount of the 4-hydroxycinnamic acid compounds and the metal salt to be used, the generation reaction proceeds by heating in any case.

In particular, in the case of the reaction under alkaline conditions, the raw material solution is adjusted to be alkaline. For example, the pH may be adjusted by adding a pH adjuster after preparing the raw material solution or the pH of a solvent to be used for the raw material solution may be adjusted in advance. When the pH is finally 7.0 or more, the generation reaction proceeds. However, when the pH exceeds 13.0, a generation reaction of another compound and decomposition of the target compound occur simultaneously with the generation reaction, accordingly the final recovery amount of the phenolic polymerizable compound decreases. Accordingly, when the reaction is performed under alkaline conditions in the production process of the invention, the pH of the raw material solution during heating for starting the generation reaction is desirably adjusted to 7.0 to 13.0.

The pH adjuster which can be used for adjusting the raw material solution to be alkaline is not particularly limited. From the viewpoint of safety, good generation efficiency, and low cost, sodium hydroxide, potassium hydroxide, sodium hydrogencarbonate, and the like are preferable. In particular, sodium hydrogencarbonate is more preferable because even when a large amount of sodium hydrogencarbonate is added, the pH of the raw material solution does not exceed 13.0. When suppressing a change in the pH of the raw material solution during the generation reaction as much as possible, various kinds of buffer solutions may be used.

When performing the reaction under acidic conditions in the production process of the invention, the raw material solution is acidic (pH of lower than 7) because the 4-hydroxycinnamic acid compounds are contained. Therefore, it is not particularly necessary to adjust the pH. However, when using one containing ingredients other than the 4-hydroxycinnamic acid compounds as the raw materials, a known pH adjuster, such as hydrochloric acid, may be used as required.

Next, the raw material solution is heated in the presence of a metal salt. By the heating treatment, the generation reaction of the target phenolic polymerizable compound is performed. In order to efficiently advance the generation reaction, the heating temperature of the raw material solution is preferably adjusted to 90° C. or higher. Considering the boiling point of the solvent to be used, it is preferable to perform pressurization and heating. It is preferable to heat the solution in such a manner that the solution temperature at least partially reaches 90° C. or higher by, for example, placing the raw material solution in an open container, and then heating the container at a high temperature exceeding the boiling point of the solvent, placing the raw material solution in an airtight container, heating the container, and performing pressurization and heating using a retort apparatus or an autoclave, and the like. It is still more preferable that the solution temperature uniformly becomes 90° C. to 150° C. in terms of increasing the generation efficiency and the recovery efficiency of the target phenolic polymerizable compound. The heating time is not limited similarly as the heating temperature and the time conditions may be determined in such a manner that the target reaction efficiently proceeds. In particular, the heating time depends on the balance of the heating temperature and the solvent amount. The heating time is desirably set according to the heating temperature and the solvent amount. For example, when the heating is performed around 130° C., the heating is preferably performed for 5 minutes to 24 hours after the solution temperature reaches 130° C. The heating may be performed once or may be repeatedly performed in a plurality of stages. When the heating is performed in a plurality of stages, it is preferable to perform the heating after newly adding only a solvent or a solvent containing a metal salt.

The completion of the generation reaction of the phenolic polymerizable compound by the heating may be judged, for example by confirming the generation amount of the phenolic polymerizable compound by the componential analysis by HPLC described in Examples below.

The phenolic polymerizable compound manufactured by the production process of the invention is a compound represented by Formula (1);

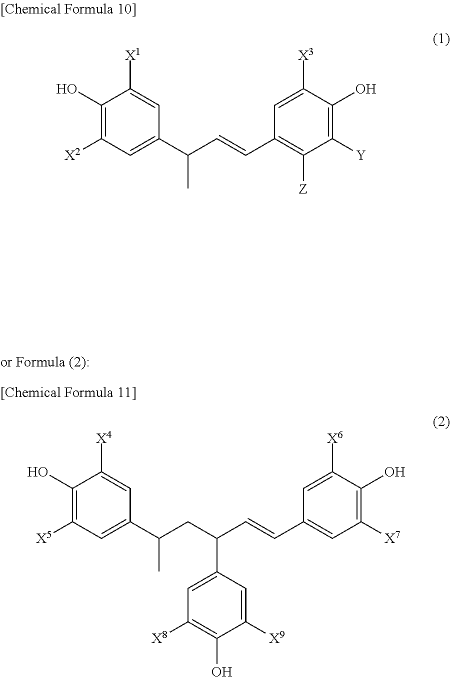

(in Formulae (1) to (2), X¹-X⁹ independently represent a hydrogen atom, a hydroxy group, a saturated or unsaturated linear or branched alkoxy group having 1-10 carbon atoms, or a saturated or unsaturated linear or branched alkyl group having 1-10 carbon atoms; Y represents a hydrogen atom, a hydroxy group, a saturated or unsaturated linear or branched alkoxy group having 1-10 carbon atoms, a saturated or unsaturated linear or branched alkyl group having 1-10 carbon atoms, or a group represented by Formula (3):

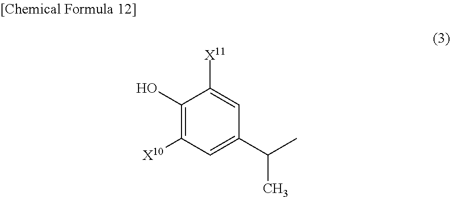

(in Formula (3), X¹⁰ and X¹¹ independently represent a hydrogen atom, a hydroxy group, a saturated or unsaturated linear or branched alkoxy group having 1-10 carbon atoms, or a saturated or unsaturated linear or branched alkyl group having 1-10 carbon atoms); and Z represents a hydrogen atom or a group represented by Formula (3); in which X¹-X¹¹ may be the same or different from one another).

Specifically, the compound represented by Formula (1) includes compounds represented by Formula (5):

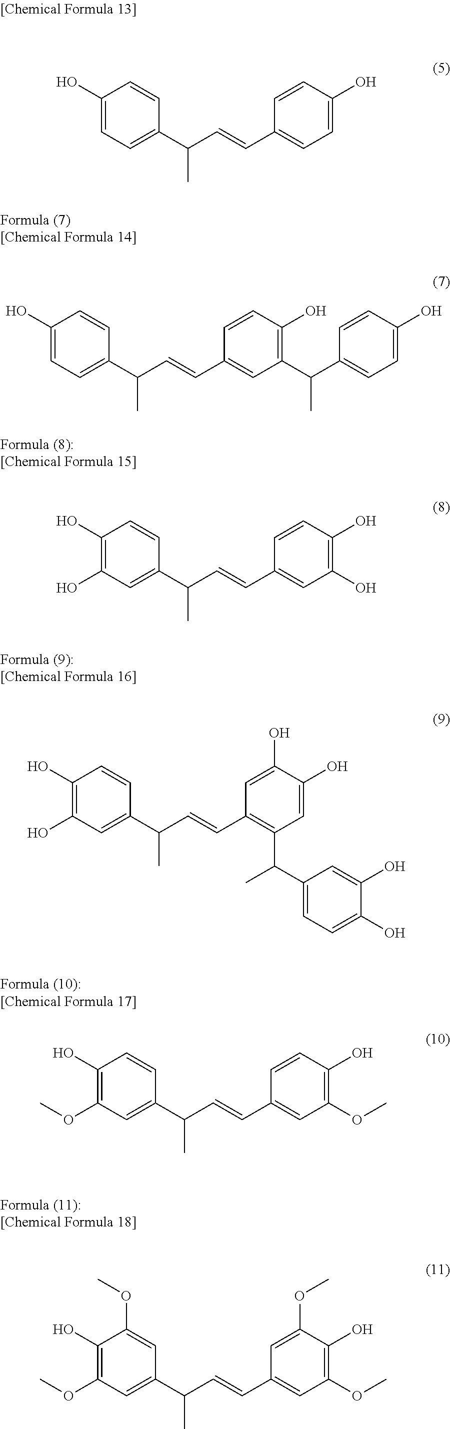

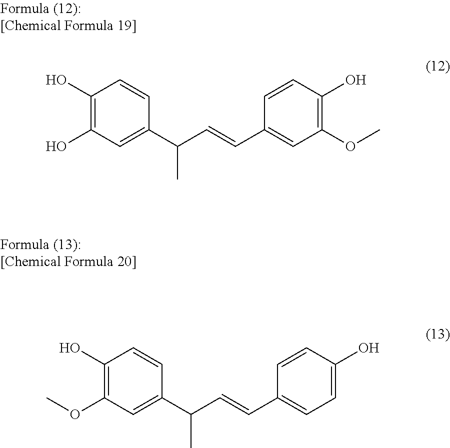

The compound represented by Formula (2) includes compounds represented by Formula (6):

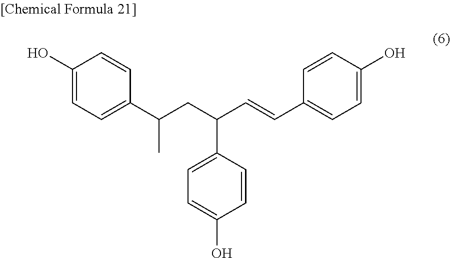 [SMILES: CC(CC(/C=C/c1ccc(O)cc1)c1ccc(O)cc1)c1ccc(O)cc1]

The phenolic polymerizable compound having physiological activity generated by the production process of the invention includes pharmacologically permissible salts.

The pharmacologically permissible salts include, for example, alkaline metal salts, such as lithium salt, sodium salt, and potassium salt alkaline earth metal salts, such as magnesium salt, calcium salt, and barium salt; aluminum salt; metal hydroxide salts, such as aluminum hydroxide salt amine salts, such as alkyl amine salt, dialkylamine salt, trialkylamine salt, alkylene diamine salt, cycloalkyl amine salt, aryl amine salt, aralkyl amine salt, and heterocyclic amine salt; amino acid salts, such as α-amino acid salt and ω-amino acid salt; peptide salt or primary, secondary, tertiary, or quaternary amine salts derived therefrom. These pharmacologically permissible salts can be used singly or as a mixture of two or more kinds thereof.

As processes for efficiently producing the phenolic polymerizable compounds represented by Formulae (5) to (13), the following processes are given.

[1] By heating p-coumaric acid in the presence of a metal salt (preferably alkaline), the compound represented by Formula (5), (6), or (7) can be produced.
[2] By heating coffeic acid in the presence of a metal salt (preferably acidic), the compound represented by Formula (8) or (9) can be produced.
[3] By heating ferulic acid in the presence of a metal salt (preferably alkaline), the compound represented by Formula (10) can be produced.
[4] By heating sinapic acid in the presence of a metal salt (preferably acidic), the compound represented by Formula (11) can be produced.
[5] By heating coffeic acid and ferulic acid in the presence of a metal salt (preferably acidic), the compound represented by Formula (12) can be produced.
[6] By heating p-coumaric acid and ferulic acid in the presence of a metal salt (preferably alkaline), the compound represented by Formula (13) can be produced.

When the phenolic polymerizable compound is produced in the process only using safe materials, the phenolic polymerizable compound can be used for foods, pharmaceutical agents, quasi-drugs, or cosmetics in a state of a mixture containing the phenolic polymerizable compound. For example, when naturally-derived 4-hydroxycinnamic acid compounds are dissolved in a hydrous ethanol solvent, and heated using sodium bicarbonate, mineral water, or mineral premix, the liquefied reactant to be obtained can be used as one of food raw materials.

When an improvement of flavor and higher functionality are desired, the concentration of the phenolic polymerizable compound is increased by condensing the reactant or a pure article of the phenolic polymerizable compound can be obtained by purifying the reactant. The condensing and the purification can be carried out by known methods. For example, the phenolic polymerizable compound can be condensed by extracting by methods such as solvent extraction methods with chloroform, ethyl acetate, ethanol, methanol, and the like, a supercritical extraction method with carbon dioxide gas, and the like. Moreover, the condensing and the purification can be also carried out utilizing column chromatography. For the condensing and the purification, a recrystallization method and a membrane treatment method with an ultrafiltration membrane or the like can be also used.

When isolating the phenolic polymerizable compound from the reactant and collecting the same, column chromatography, high performance liquid chromatography (HPLC), and the like may be used.

By subjecting the condensed substance or the purified substance to drying under reduced pressure or freeze-drying as required to remove the solvent, a powdery solid can be obtained.

A further effect and efficacy of the phenolic polymerizable compound obtained by the invention can be used in the range which is analogized from the obtained physiological activity data.

Since the safety of the 4-hydroxycinnamic acid compounds which are the raw materials to be used in the invention and also a solvent, a metal salt, and a pH adjuster to be used for the synthesis are already generally confirmed, it is considered that the safety of the phenolic polymerizable compound obtained by the invention is also similarly excellent.

The phenolic polymerizable compound has one or more physiological activities, such as anti-cancer activity, anti-cancer activity to oral cancer, and lipase inhibition activity, as described in Examples described later.

Accordingly, the phenolic polymerizable compound can be used as an active ingredient of anticancer agents or anticancer agents for oral cancer and lipase inhibitors. Moreover, the phenolic polymerizable compound having lipase inhibition activity can be used as an active ingredient of anti-obesity agents or skin disease therapeutic agents.

The phenolic polymerizable compound can be compounded in foods, pharmaceutical agents, quasi-drugs, cosmetics, and the like for use.

The foods may be in any form, such as beverages, alcoholic beverages, jellies, confectioneries, and the like, for example. Among the confectionaries, a hard candy, a soft candy, a gummi candy, a tablet, and the like which are excellent in storageability or portability from the capacity and the like are given as examples but the confectionaries are not particularly limited thereto. The foods also include functional foods, health foods, health-conscious foods, and the like.

The pharmaceutical agents include solid preparations, such as powder agents, tablets, pills, capsule agents, fine grain agents, and granule agents; liquid agents, such as water agents, suspension agents, and emulsion agents; gel agents, and the like. The tablets, pills, granule agents, and granules in capsule agents may be sugar-coated with sugars, such as sucrose, and sugar alcohols, such as maltitol, coated with gelatin, hydroxypropyl cellulose, hydroxypropyl methylcellulose, and the like, or covered with a film of a gastric-soluble substance or an enteric-soluble substance, as required. The pharmaceutical agents mentioned above can be also subjected to known solubilization treatment in order to increase the solubility of the pharmaceuticals. The liquid agents may be compounded in injection agents and drop agents for use based on a usual method.

The quasi-drugs include are toothpastes, mouthwashes, mouse rinses, nutrients, and the like.

The cosmetics include lotions, milky lotions, creams, facial mask agents, finishing cosmetics, hair care products, face washing agents, bath agents, antiperspirants, and the like. With respect to the cosmetics, a recovery from pimples is expected from the lipase inhibition effect, and the cosmetics can be utilized for the purpose of prevention of pimples and recovery from pimples.

When preparing foods, pharmaceutical agents, quasi-drugs, or cosmetics using the phenolic polymerizable compound, ingredients usually used for foods, pharmaceutical agents, quasi-drugs, or cosmetics can be compounded as appropriate in the range where the effects of the invention are not impaired.

For example, in the case of foods, the phenolic polymerizable compound can be combined with raw materials or materials usually compounded in foods, such as water, alcohol, starch, protein, fiber, sugar, lipid, vitamin, mineral, a flavoring agent, a colorant, a sweetener, a seasoning, a stabilizer, and an antiseptic agent.

In the case of pharmaceutical agents, quasi-drugs, or cosmetics, the phenolic polymerizable compound is combined with a main agent, a base material, a surfactant, a foaming agent, a moisturizing agent, a thickening agent, a clearing agent, a flavoring agent, a colorant, a stabilizer, an antiseptic agent, a disinfectant, and the like, and can be prepared into the form of a liquid, an ointment, the final form which can be spray ejected, and the like based on a usual method.

When adding the phenolic polymerizable compound to foods, it is usually preferable to add the same into the foods in a proportion of 0.001 to 20% by weight.

When the phenolic polymerizable compound is used for medical application, the intake, for example, is not particularly limited insofar as a desired improvement effect, a desired medical treatment effect, or a desired preventive effect is obtained and is usually selected as appropriate according to the aspect thereof, the age, sex, physical constitution, other conditions of patients, and the kind and extent of diseases, and the like. The intake may be about 0.1 mg to about 1,000 mg per day and can be divided into 1 to 4 doses per day.

When adding the phenolic polymerizable compound to quasi-drugs or cosmetics, it is usually preferable to add the same into the quasi-drugs or the cosmetics in a proportion of 0.001 to 30% by weight.

Since the phenolic polymerizable compound is excellent in safety, the phenolic polymerizable compound may be used not only for human beings and may be compounded in therapeutic agents or feed for nonhuman animals, such as mammals, such as rats, mice, guinea pigs, rabbits, sheep, pigs, cows, horses, cats, dogs, apes, and chimpanzees, birds, amphibians, and reptiles. The feed includes, for example, cattle feeds for sheep, pigs, cows, horses, chickens, and the like, feeds for small animals for rabbits, rats, mice, and the like, feeds for fish and shellfishes for eels, sea breams, yellowtails, shrimps, and the like, and pet foods for dogs, cats, caged little birds, squirrels, and the like.

Next, the invention is described in detail with reference to Examples but the invention is not limited only to the Examples.

### EXAMPLES

### Example 1

### Generation of Phenolic Polymerizable Compound from p-coumaric Acid

A mixed liquid (pH=7.5) obtained by dissolving 500 mg of p-coumaric acid (Wako Pure Chemical Industries, Ltd.) in 10 ml of ethanol, and then adding 10 ml of an aqueous 5% sodium hydrogencarbonate solution (Wako Pure Chemical Industries, Ltd.) was heated at 130° C. for 40 minutes in an autoclave (“SANYO LABO AUTOCLAVE” manufactured by SANYO Electric Co., Ltd., which was used in the following Examples). 1 ml of the obtained reactant was diluted with methanol in a measuring cylinder to 50 ml. Then, 10 μl of the resultant reactant was analyzed by HPLC.

The HPLC analysis was performed under the following conditions.

Column: Negative-phase column “Develosil (Registered Trademark) C-30-UG-5” (4.6 mmi.d.×250 mm)
Mobile phase: A . . . H₂O (0.1% trifluoroacetic acid (TFA)), B . . . Acetonitrile (0.1% TFA)
Flow velocity: 1 ml/min

### Pouring: 10 μl

### Detection: 254 nm

Gradient (% by capacity): From 80% A/20% B to 20% A/80% B for 30 minutes, From 20% A/80% B to 100% B for 5 minutes, 100% B for 10 minutes (all straight line)

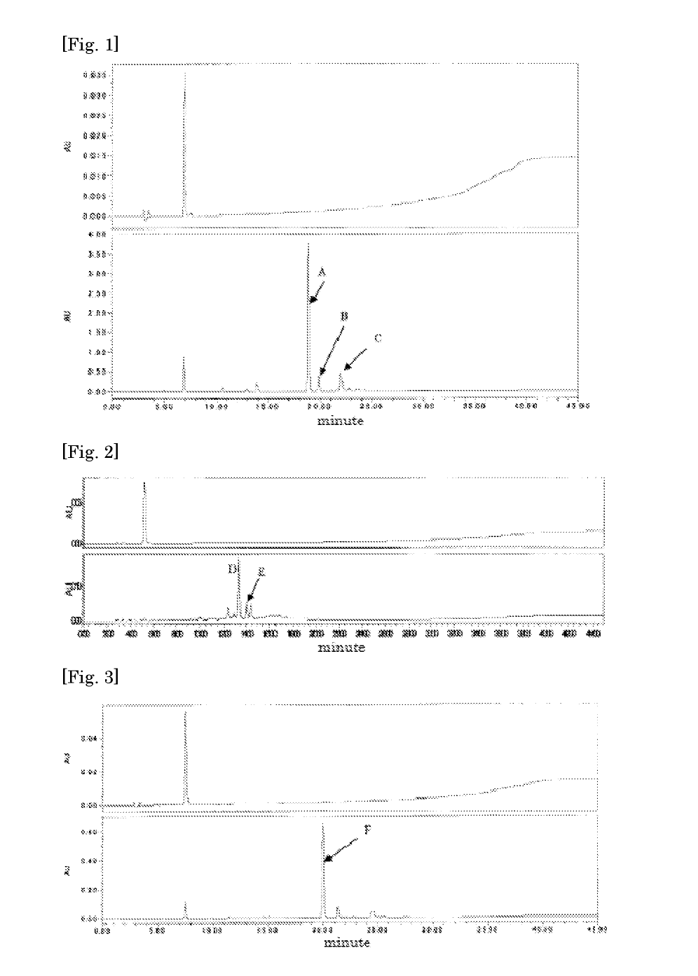

The obtained chromatograms are shown in FIG. 1. The upper view shows the chromatogram of p-coumaric acid and the lower view shows the chromatogram after the reaction. The p-coumaric acid decreased due to the reaction and some peaks at which the p-coumaric acid increased were confirmed. Therefore, it was confirmed that a plurality of compounds were generated including the peaks of A, B, and C.

### Example 2

### Isolation and Structural Determination of Phenolic Polymerizable Compound

The compounds contained in the peaks shown by A, B, and C of FIG. 1 in the reactants obtained in Example 1 were isolated by fractionation HPLC. When the isolated HPLC eluate was dried according to a usual method, 115 mg of a yellow powdery phenolic polymerizable compound (hereinafter referred to as UHA7009) was obtained from A. 15 mg of a yellow powdery phenolic polymerizable compound (hereinafter referred to as UHA7010) was obtained from B. 32 mg of a yellow powdery phenolic polymerizable compound (hereinafter referred to as UHA7011) was obtained from C.

Subsequently, when the molecular weight of each of the UHA7009, the 7010, and the 7011 was measured using a high resolution electron ionization-mass spectrometry (hereinafter referred to as EI-MS), the measured values were UHA7009: 240.2973, UHA7010: 360.4455, and UHA7011: 360.4458. The following molecular formulae were obtained from the comparison with the theoretical values.

### UHA7009

Theoretical value C16H16O2 (M⁺): 240.2970
Molecular formula C₁₆H₁₆O₂

### UHA7010

Theoretical value C24H24O3 (M⁺): 360.4456
Molecular formula C₂₄H₂₄O₃

### UHA7011

Theoretical value C24H24O3 (M⁺): 360.4456
Molecular formula C₂₄H₂₄O₃

Next, the UHA7009, the UHA7010, and the UHA7011 were subjected to nuclear magnetic resonance (NMR) measurement. Then, it was confirmed that the UHA7009 had the structure represented by Formula (5), the UHA7010 had the structure represented by Formula (6), and the UHA7011 had the structure represented by Formula (7) from the analysis of 1H-NMR, 13C-NMR, and various two-dimensional NMR data. This shows that the phenolic polymerizable compounds represented by Formulae (5) to (7) can be efficiently generated by the process of the invention.

With respect to the NMR measured values, when the sites of the carbon atoms of the UHA7009, the UHA7010, and the UHA7011 represented by Formulae (5) to (7) are as follows, the 1H nuclear magnetic resonance spectrum and the 13C nuclear magnetic resonance spectrum of each site of the UHA7009, the UHA7010, and the UHA7011 are shown in Tables 1, 2, and 3, respectively.

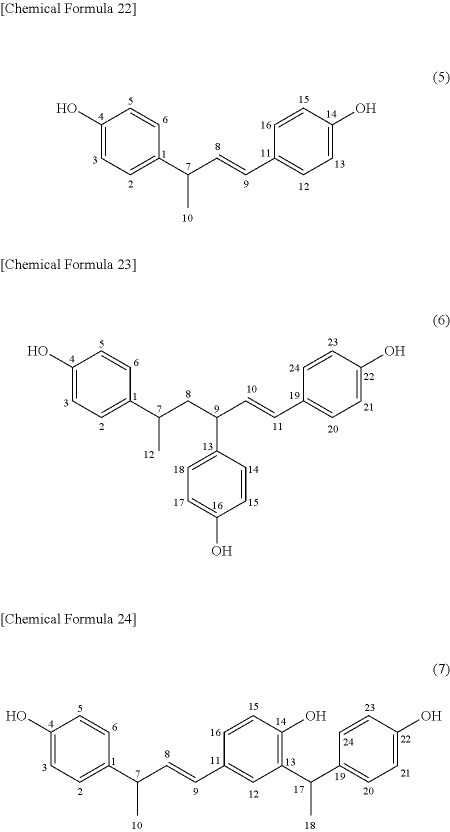 [SMILES: CC(/C=C/c1ccc(O)cc1)c1ccc(O)cc1 ; CC(CC(/C=C/c1ccc(O)cc1)c1ccc(O)cc1)c1ccc(O)cc1 ; CC(/C=C/c1ccc(O)c(C(C)c2ccc(O)cc2)c1)c1ccc(O)cc1]

The values in the tables are δ and ppm and are values measured with methanol-d3.

TABLE 1
NMR Data (UHA7009)

| | UHA7009 | |
|---|---|---|
| Site | 13C | 1H |
| 1 | 138.2 | |
| 2, 6 | 128.1 | 7.05 (2H, d, J = 8.7 Hz) |
| 3, 5 | 116.1 | 6.72 (2H, d, J = 8.7 Hz) |
| 4 | 156.4 | |
| 7 | 42.9 | 3.46 (1H, dd-like) |
| 8 | 133.9 | 6.14 (1H, dd, J = 6.4, 16.0 Hz) |
| 9 | 128.8 | 6.26 (1H, d, J = 16.0 Hz) |
| 10 | 22.1 | 1.36 (3H, d, J = 6.9 Hz) |
| 11 | 130.8 | |
| 12, 16 | 129.1 | 7.17 (2H, d, J = 8.2 Hz) |
| 13, 15 | 116.2 | 6.74 (2H, d, J = 8.2 Hz) |
| 14 | 157.5 | |

TABLE 2
NMR Data (UHA7010)

| | UHA7010 | |
|---|---|---|
| Site | 13C | 1H |
| 1 | 139.3 | |
| 2, 6 | 129.0 | 6.94 (2H, m) |
| 3, 5 | 116.2 | 6.70 (2H, m) |
| 4 | 156.7 | |
| 7 | 37.9 | 2.62 (1H, dd-like) |
| 8 | 46.0 | 1.94 (1H, dd-like) |
| 9 | 47.4 | 3.11 (1H, dd-like) |
| 10 | 132.6 | 6.07 (1H, dd, J = 6.0, 15.5 Hz) |
| 11 | 129.3 | 6.10 (1H, d, J = 15.5 Hz) |
| 12 | 23.5 | 1.19 (3H, d, J = 6.9 Hz) |
| 13 | 136.6 | |
| 14, 18 | 129.1 | 6.95 (2H, m) |
| 15, 17 | 116.1 | 6.71 (2H, m) |
| 16 | 156.5 | |
| 19 | 130.8 | |
| 20, 24 | 128.3 | 7.16 (2H, d, J = 8.7 Hz) |
| 21, 23 | 116.2 | 6.66 (2H, d, J = 8.7 Hz) |
| 22 | 157.7 | |

TABLE 3
NMR Data (UHA7011)

| | UHA7011 | |
|---|---|---|
| Site | 13C | 1H |
| 1 | 138.1 | |
| 2, 6 | 128.3 | 7.05 (2H, d, J = 8.7 Hz) |
| 3, 5 | 115.8 | 6.68 (2H, m) |
| 4 | 156.2 | |
| 7 | 43.2 | 3.43 (1H, dd-like) |
| 8 | 134.2 | 6.10 (1H, d, J = 8.3, 16.0 Hz) |
| 9 | 128.9 | 6.20 (1H, d, J = 16.0 Hz) |
| 10 | 22.1 | 1.31 (1H, d, J = 8.7 Hz) |
| 11 | 131.0 | |
| 12 | 127.8 | 6.92 (1H, s) |
| 13 | 134.8 | |
| 14 | 154.9 | |
| 15 | 125.3 | 6.88 (1H, d, J = 8.3 Hz) |
| 16 | 126.9 | 7.14 (1H, d, J = 8.3 Hz) |
| 17 | 37.9 | 4.44 (1H, d-like) |
| 18 | 21.5 | 1.50 (1H, d, J = 7.3 Hz) |
| 19 | 138.8 | |
| 20, 24 | 129.7 | 7.07 (2H, d, J = 8.7 Hz) |
| 21, 23 | 116.2 | 6.70 (2H, m) |
| 22 | 156.6 | |

The physicochemical properties of the UHA7009, the UHA7010, and the UHA7011 were as shown in Table 4.

TABLE 4

| Compound | UHA7009 | UHA7010 | UHA7011 |
|---|---|---|---|
| Property | Yellow | Yellow | Yellow |
| Solubility | powder | powder | powder |
| Water | Poorly soluble | Poorly soluble | Poorly soluble |
| Methanol | Soluble | Soluble | Soluble |
| Ethanol | Soluble | Soluble | Soluble |
| DMSO | Soluble | Soluble | Soluble |
| Chloroform | Soluble | Soluble | Soluble |
| Ethyl acetate | Soluble | Soluble | Soluble |

### Example 3

### Examination of Metal Salt to Generation Amount of Phenolic Polymerizable Compound

In order to examine metal salts capable of efficiently generating the three kinds of the phenolic polymerizable compounds, 50 mg of p-coumaric acid, 1 ml of ethanol, 1 ml of deionized water (1 ml of mineral water in the case of mineral water containing metal salt), and 50 mg of metal salt were added, and then heated at 130° C. for 30 minutes in an autoclave. All of the UHA7009, the UHA7010, and the UHA7011 were confirmed in the obtained reactants. The amount (% by weight) of the UHA7009 whose generation amount was the highest is shown in Table 5. Only the deionized water is the control. The metal salts used were sodium hydrogencarbonate, sodium carbonate, sodium hydroxide, sodium dihydrogenphosphate, sodium chloride, calcium chloride, calcium phosphate, calcium lactate, calcium carbonate, potassium hydroxide, potassium chloride, potassium dihydrogenphosphate, magnesium phosphate, magnesium chloride, magnesium sulfate, barium carbonate, lithium carbonate, zinc sulfate, copper chloride, iron ammonium citrate, strontium carbonate (all manufactured by Wako Pure Chemical Industries, Ltd.), mineral water (Trade name “Gerolsteiner”, manufactured by SAPPORO Beverage, Ltd.), and mineral premix.

TABLE 5

| | pH | UHA7009 | | pH | UHA7009 |
|---|---|---|---|---|---|
| | (Before reaction) | (% by weight) | | (Before reaction) | (% by weight) |
| Water (Deionized | 3.6 | 0.17 | Potassium | 4.2 | 3.06 |
| water) | | | dihydrogenphosphate | | |
| Sodium | 7.2 | 22.00 | Magnesium phosphate | 5.1 | 0.51 |
| hydrogencarbonate | | | | | |
| Sodium carbonate | 10.2 | 20.88 | Magnesium chloride | 3.3 | 2.55 |
| Sodium hydroxide | 13.8 | 0.04 | Magnesium sulfate | 3.9 | 0.82 |
| Sodium | 4.5 | 3.01 | Barium carbonate | 5.0 | 0.86 |
| dihydrogenphosphate | | | | | |
| Sodium chloride | 3.4 | 1.61 | Lithium carbonate | 8.1 | 23.50 |
| Calcium chloride | 3.2 | 4.08 | Zinc sulfate | 3.7 | 12.12 |
| Calcium phosphate | 4.5 | 18.89 | Copper chloride | 2.6 | 6.63 |
| Calcium lactate | 5.0 | 0.99 | Iron ammonium citrate | 4.7 | 0.58 |
| Calcium carbonate | 5.1 | 0.58 | Strontium carbonate | 5.2 | 0.47 |
| Potassium hydroxide | 14.0 | 0.04 | Mineral water | 5.0 | 0.35 |
| | | | (Gerolsteiner) | | |
| Potassium chloride | 3.6 | 2.29 | Mineral premix | 5.2 | 0.69 |

As a result, when the metal salts other than sodium hydroxide and potassium hydroxide were used, the generation amount of the UHA7009 was twice or more the amount when deionized water was used.

In particular, when sodium hydrogencarbonate, sodium carbonate, and lithium carbonate in which the raw material solution exhibited alkalinity were used, the generation amount of the UHA7009 was 20 times or more the amount when deionized water was used.

When sodium hydroxide and potassium hydroxide were used, the pH of the mixed liquid during the reaction exceeded 13. Therefore, it is considered that another reaction and a polymerization reaction occur, and as a result, the generation amount of the UHA7009 decreased.

### Example 4

### Difference in Generation Amount of 4-vinyl Phenolic Polymerizable Compound Depending on Heating Temperature

A mixed solution (pH=7.5) of 50 mg of p-coumaric acid, 1 ml of ethanol, and 1 ml of an aqueous 5% sodium hydrogencarbonate solution was heated for 20 minutes in an autoclave at each of the temperature conditions (70° C., 90° C., 110° C., and 130° C.). 1 ml of the reactants obtained at each temperature condition was diluted with methanol in a measuring cylinder to 50 ml, and then analyzed by HPLC in the same manner as in Example 1.

As a result, the presence of the three kinds of the UHA7009, the UHA7010, and the UHA7011 were confirmed under the reaction condition of 90° C. or higher. Among the above, only the UHA7009 whose generation amount was large is described. With respect to the generation ratio of the UHA7009 from p-coumaric acid, the UHA7009 was not generated at 70° C., the generation amount thereof was very slight at 90° C., the generation amount thereof was 5% by weight at 110° C., and the generation amount was 22% by weight at 130° C. More specifically, the heating at 130° C. was the most efficient.

When further increasing the reaction temperature and the temperature exceeds about 150° C., the generation ratio decreased. Accordingly, it was found that the heating temperature is preferably in the range of 90° C. to 150° C. from the viewpoint of the recovery efficiency of the phenolic polymerizable compound.

### Example 5

### Generation of Phenolic Polymerizable Compound from Coffeic Acid

A mixed liquid (pH=5.4) obtained by dissolving 1 g of coffeic acid (Wako Pure Chemical Industries, Ltd.) in 20 ml of ethanol, and then adding 20 ml of mineral water was heated at 130° C. for 80 minutes in total in an autoclave. 1 ml of the obtained reactant was diluted with methanol in a measuring cylinder to 50 ml. Then, the HPLC analysis was performed.

The HPLC analysis was performed in the same manner as in Example 1.

The obtained chromatograms are shown in FIG. 2. The upper view shows the chromatogram of coffeic acid and the lower view shows the chromatogram after the heating. It was confirmed that a plurality of compounds were generated including the peaks of D and E.

### Example 6

### Isolation and Structural Determination of Phenolic Polymerizable Compound

The compounds contained in the peaks shown by D and E of FIG. 2 in the reactants obtained in Example 5 were isolated by fractionation HPLC. When the isolated HPLC eluate was dried according to a usual method, 30 mg of a brown powdery phenolic polymerizable compound (hereinafter referred to as UHA6005) was obtained from the peak of D. 10 mg of a novel brown powdery compound (hereinafter referred to as UHA6006) was obtained from the peak E.

Subsequently, when the molecular weight of each of the UHA6005 and the UHA6006 was measured by a high resolution EI-MS, the measured values were UHA6005: 272.2955 and UHA6006: 408.4440. The following molecular formulae were obtained from the comparison with the theoretical values.

### UHA6005

Theoretical value C16H16O4 (M⁺): 272.2958
Molecular formula C₁₆H₁₆O₄

### UHA6006

Theoretical value C24H24O6 (M⁺): 408.4438
Molecular formula C₂₄H₂₄O₆

Next, the UHA6005 and the UHA6006 were subjected to nuclear magnetic resonance (NMR) measurement. Then, it was confirmed that the UHA6005 had the structure represented by Formula (8) and the UHA6006 had the structure represented by Formula (9) from the analysis of 1H-NMR, 13C-NMR, and various two-dimensional NMR data. This shows that the phenolic polymerizable compounds represented by Formulae (8) and (9) can be efficiently generated by the process of the invention. With respect to the NMR measured values, when the sites of the UHA6005 and the UHA6006 represented by Formulae (8) and (9) are as follows, the 1H nuclear magnetic resonance spectrum and the 13C nuclear magnetic resonance spectrum of each site of the UHA6005 and the UHA6006 are shown in Tables 6 and 7, respectively.

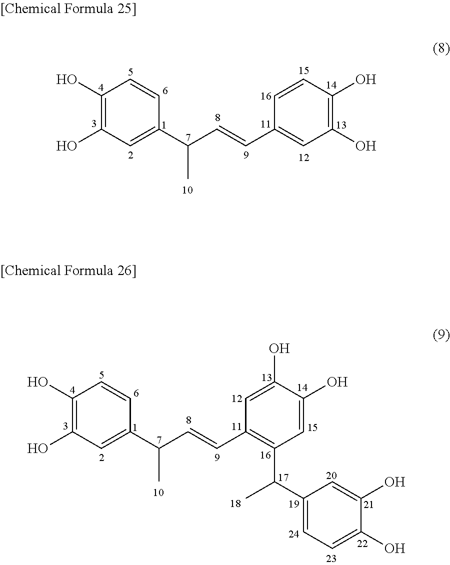 [SMILES: CC(/C=C/c1ccc(O)c(O)c1)c1ccc(O)c(O)c1 ; CC(/C=C/c1cc(O)c(O)cc1C(C)c1ccc(O)c(O)c1)c1ccc(O)c(O)c1]

The values in the tables are δ and ppm and are values measured with methanol-d3.

TABLE 6

| | UHA6005 | |
|---|---|---|
| Site | 13C | 1H |
| 1 | 139.2 | |
| 2 | 115.4 | 6.68 (1H, m) |
| 3 | 144.4 | |
| 4 | 146.3 | |
| 5 | 116.3 | 6.66 (1H, d, J = 8.3 Hz) |
| 6 | 119.4 | 6.55 (1H, dd, J = 2.3, 8.3 Hz) |
| 7 | 43.1 | 3.40 (1H, dd-like) |
| 8 | 133.9 | 6.09 (1H, dd, J = 6.4, 16.0 Hz) |
| 9 | 129.1 | 6.19 (1H, d, J = 16.0 Hz) |
| 10 | 22.1 | 1.34 (3H, d, J = 6.9 Hz) |
| 11 | 131.6 | |
| 12 | 113.6 | 6.80 (1H, s) |
| 13 | 146.1 | |
| 14 | 145.7 | |
| 15 | 116.3 | 6.69 (1H, d, J = 8.3 Hz) |
| 16 | 119.4 | 6.66 (1H, d, J = 8.3 Hz) |

TABLE 7
NMR Data

| | UHA6006 | |
|---|---|---|
| Site | 13C | 1H |
| 1 | 139.0 | |
| 2 | 115.2 | 6.63 (1H, m) |
| 3 | 144.2 | |
| 4 | 146.1 | |
| 5 | 116.1 | 6.65 (1H, m) |
| 6 | 119.7 | 6.48 (1H, m) |
| 7 | 43.2 | 3.36 (1H, dd-like) |
| 8 | 136.2 | 5.90 (1H, dd, J = 6.4, 15.6 Hz) |
| 9 | 127.1 | 6.49 (1H, d, J = 15.6 Hz) |
| 10 | 21.8 | 1.29 (3H, d, J = 6.9 Hz) |
| 11 | 129.7 | |
| 12 | 114.1 | 6.80 (1H, s) |
| 13 | 144.3 | |
| 14 | 145.5 | |
| 15 | 115.4 | 6.62 (1H, m) |
| 16 | 136.8 | |
| 17 | 40.7 | 4.11 (1H, d, J = 7.3 Hz) |
| 18 | 22.8 | 1.44 (3H, d, J = 7.3 Hz) |
| 19 | 140.1 | |
| 20 | 116.0 | 6.54 (1H, d, J = 2.3 Hz) |
| 21 | 144.0 | |
| 22 | 145.9 | |
| 23 | 116.3 | 6.68 (1H, d, J = 8.2 Hz) |
| 24 | 120.0 | 6.45 (1H, dd, J = 2.3, 8.2 Hz) |

The physicochemical properties of the UHA6005 and the UHA6006 were as shown in Table 8.

TABLE 8

| Compound | UHA6005 | UHA6006 |
|---|---|---|
| Property | Brown | Brown |
| Solubility | powder | powder |
| Water | Poorly soluble | Poorly soluble |
| Methanol | Soluble | Soluble |
| Ethanol | Soluble | Soluble |
| DMSO | Soluble | Soluble |
| Chloroform | Soluble | Soluble |
| Ethyl acetate | Soluble | Soluble |

### Example 7

### Generation of Phenolic Polymerizable Compound from Ferulic Acid

A mixed liquid (pH=7.3) obtained by dissolving 500 mg of ferulic acid (Wako Pure Chemical Industries, Ltd.) in 10 mL of ethanol and then adding 10 mL of an aqueous 5% sodium hydrogencarbonate solution was heated at 130° C. for 40 minutes in an autoclave. 1 mL of the obtained reactant was diluted with methanol in a measuring cylinder to 50 mL. Then, 10 μL of the resultant reactant was analyzed by HPLC under the same conditions as those of Example 1.

The obtained chromatograms are shown in FIG. 3. The upper view shows the chromatogram before the generation reaction and the lower view shows the chromatogram after the generation reaction. In the upper view, the peak of ferulic acid is shown. As shown in the lower view, it was confirmed that the ferulic acid decreased due to the generation reaction and a plurality of compounds were generated including the peak F.

### Example 8

### Isolation and Structural Determination of Phenolic Polymerizable Compound

The compounds contained in the peak shown by F of FIG. 3 in the reactants obtained in Example 7 were isolated by fractionation HPLC. When the isolated HPLC eluate was dried according to a usual method, 301 mg of a yellow powdery compound (hereinafter referred to as UHA7004) was obtained.

Subsequently, when the molecular weight of the UHA7004 was measured by a high resolution EI-MS, the measured value was UHA7004: 300.3495. The following molecular formula was obtained from the comparison with the theoretical value.

### UHA7004

Theoretical value C18H20O4 (M⁺): 300.3490
Molecular formula C₁₈H₂₀O₄

Next, the UHA7004 was subjected to NMR measurement. Then, it was confirmed that the UHA7004 had the structure represented by Formula (10) from the analysis of 1H-NMR, 13C-NMR, and various two-dimensional NMR data. This shows that the phenolic polymerizable compound represented by Formula (10) can be efficiently generated by the process of the invention.

With respect to the NMR measured values, when the sites of the UHA7004 represented by Formula (10) are as follows, the 1H nuclear magnetic resonance spectrum and the 13C nuclear magnetic resonance spectrum of each site of the UHA7004 are shown in Table 9.

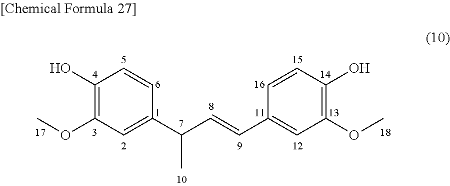

The values in the tables are δ and ppm and are values measured with methanol-d3.

TABLE 9
NMR Data (UHA7004)

| | UHA7004 | |
|---|---|---|
| | 13C | 1H |
| 1 | 139.0 | |
| 2 | 112.0 | 6.79 (1H, d, J = 2.3 Hz) |
| 3 | 149.0 | |
| 4 | 145.7 | |
| 5 | 116.1 | 6.73 (1H, d, J = 7.8 Hz) |
| 6 | 120.5 | 6.77 (1H, dd, J = 2.3, 7.8 Hz) |
| 7 | 43.4 | 3.47 (1H, dd-like) |
| 8 | 134.1 | 6.18 (1H, dd, J = 6.4, 15.5 Hz) |
| 9 | 129.3 | 6.27 (1H, d, J = 15.5 Hz) |
| 10 | 22.0 | 1.37 (3H, d, J = 6.9 Hz) |
| 11 | 131.4 | |
| 12 | 110.2 | 6.91 (1H, d, J = 1.8 Hz) |
| 13 | 148.9 | |
| 14 | 147.0 | |
| 15 | 116.1 | 6.69 (1H, d, J = 8.2 Hz) |
| 16 | 120.5 | 6.67 (1H, d, J = 1.8, 8.2 Hz) |
| 17 | 56.4 | 3.80 (3H, s) |
| 18 | 56.3 | 3.80 (3H, s) |

The physicochemical properties of the UHA7004 were as follows.

(Property)

Yellow powder

(Solubility)

Water: Poorly soluble

### Methanol: Soluble

### Ethanol: Soluble

### DMSO: Soluble

### Chloroform: Soluble

Ethyl acetate: Soluble

### Example 9

### Generation of Phenolic Polymerizable Compound from Sinapic Acid

500 mg of sinapic acid was dissolved in 10 ml of ethanol, and then 10 ml of mineral water was mixed with the solution, thereby obtaining a sinapic acid containing solution (pH=4.8). The sinapic acid containing solution was heated at 130° C. for 90 minutes in an autoclave. 1 ml of the obtained reactant solution was taken out, diluted with methanol in a measuring cylinder to 50 ml, and then analyzed by HPLC in the same manner as in Example 1.

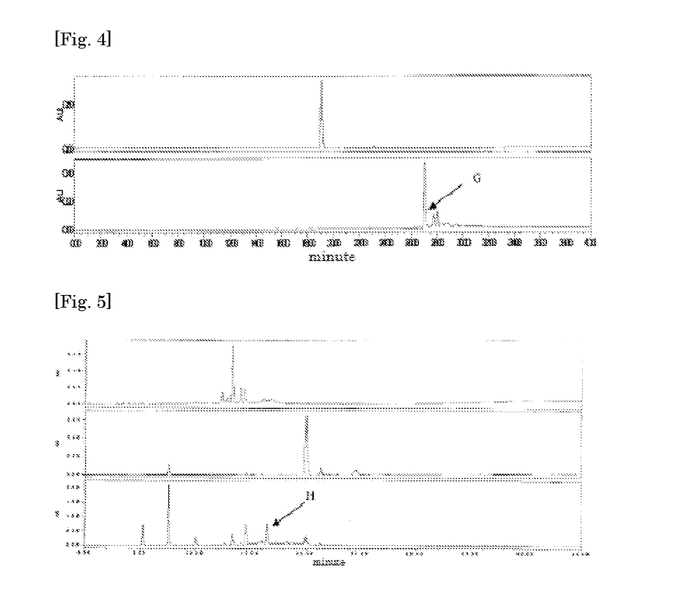

The obtained chromatograms are shown in FIG. 4. The upper view shows the chromatogram before the generation reaction and the lower view shows the chromatogram after the generation reaction. In the upper view, the peak of sinapic acid is shown. As shown in the lower view, it was confirmed that the sinapic acid decreased due to the generation reaction and a plurality of compounds were generated including the peak G.

### Example 10

### Isolation and Structural Determination of Phenolic Polymerizable Compound

The compounds contained in the peak shown by G of FIG. 4 in the reactants obtained in Example 9 were isolated by fractionation HPLC. When the isolated HPLC eluate was dried according to a usual method, 59.8 mg of a brown powdery compound (hereinafter referred to as UHA9019) was obtained.

Subsequently, when the molecular weight of the UHA9019 was measured by a high resolution EI-MS, the measured value was 360.4005, and the following molecular formula was obtained from the comparison with the theoretical value.

### UHA9019

Theoretical value C20H24O6 (M⁺): 360.4010
Molecular formula C₂₀H₂₄O₆

Next, the UHA9019 was subjected to NMR measurement. Then, it was confirmed that the UHA9019 had the structure represented by Formula (11) from the analysis of 1H-NMR, 13C-NMR, and various two-dimensional NMR data. This shows that the phenolic polymerizable compound represented by Formula (11) can be efficiently generated by the process of the invention.

With respect to the NMR measured values, when the sites of the UHA9019 represented by Formula (11) are as follows, the 1H nuclear magnetic resonance spectrum and the 13C nuclear magnetic resonance spectrum of each site of the UHA9019 are shown in Table 10.

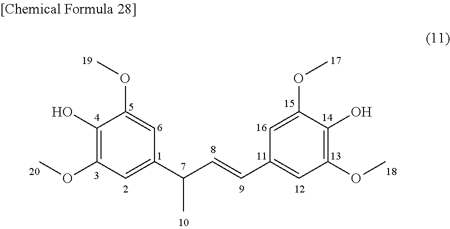

The values in the tables are δ and ppm and are values measured with methanol-d3.

TABLE 10
NMR Data (UHA9019)

| | UHA9019 | |
|---|---|---|
| Site | 13C | 1H |
| 1 | 138.1 | |
| 2, 6 | 105.7 | 6.54 (2H, s) |
| 3, 5 | 149.3 | |
| 4 | 149.1 | |
| 7 | 44.0 | 3.51 (1H, dd-like) |
| 8 | 134.4 | 6.23 (1H, dd, J = 5.9, 15.6 Hz) |
| 9 | 129.7 | 6.30 (1H, d, J = 15.6 Hz) |
| 10 | 22.0 | 1.42 (3H, d, J = 6.9 Hz) |
| 11 | 130.4 | |
| 12, 16 | 104.7 | 6.64 (2H, s) |
| 13, 15 | 149.4 | |
| 14 | 149.1 | |
| 17, 18, 19, 20 | 58.4 | 3.83 (12H, s) |

The physicochemical properties of UHA9019 were as follows.

(Property)

Brown powder

(Solubility)

Water: Poor soluble

### Methanol: Soluble

### Ethanol: Soluble

### DMSO: Soluble

### Chloroform: Soluble

Ethyl acetate: Soluble

### Example 11

### Generation of Phenolic Polymerizable Compound from Mixture of Coffeic Acid and Ferulic Acid

A mixed liquid (pH=4.3) obtained by dissolving 1 g of coffeic acid and 1 g of ferulic acid in 20 mL of ethanol, and then adding 20 mL of mineral water was heated at 130° C. for 90 minutes in an autoclave. 1 mL of the obtained reactant was diluted with methanol in a measuring cylinder to 50 mL, and then 10 μL of the resultant reactant was analyzed by HPLC under the same conditions as those of Example 1.

The obtained chromatograms are shown in FIG. 5. The upper view shows the chromatogram of the solution after the reaction of only coffeic acid, the middle view shows the chromatogram of the solution of the reactant of only ferulic acid, and the lower view shows the chromatogram after the mixture of coffeic acid and ferulic acid was generated and reacted. In the lower view, the peak H different from those of the UHA6005, the UHA6006, and the UHA7004 was observed. The compound represented by H was considered to be a product derived from both the coffeic acid and the ferulic acid.

### Example 12

### Isolation and Structural Determination of Phenolic Polymerizable Compound

The compounds contained in the peak shown by H of FIG. 5 in the reactants obtained in Example 11 were isolated by fractionation HPLC. When the isolated HPLC eluate was dried according to a usual method, 133 mg of a yellow powdery novel compound (hereinafter referred to as UHA9020) was obtained.

Subsequently, when the molecular weight of the UHA9020 was measured by a high resolution EI-MS, the measured value was 286.3220, and the following molecular formula was obtained from the comparison with the theoretical value.

### UHA9020

Theoretical value C17H18O4 (M⁺): 286.3224
Molecular formula C₁₇H₁₈O₄

Next, the UHA9020 was subjected to NMR measurement. Then, it was confirmed that the UHA9020 had the structure represented by Formula (12) from the analysis of 1H-NMR, 13C-NMR, and various two-dimensional NMR data. This shows that the novel phenolic polymerizable compound represented by Formula (12) can be efficiently generated by the process of the invention.

With respect to the NMR measured values, when the sites of the UHA9020 represented by Formula (12) are as follows, the 1H nuclear magnetic resonance spectrum and the 13C nuclear magnetic resonance spectrum of each site of the UHA9020 are shown in Table 11.

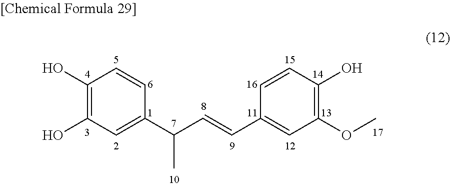 [SMILES: COc1cc(/C=C/C(C)c2ccc(O)c(O)c2)ccc1O]

The values in the table are δ and ppm and are values measured with methanol-d3.

TABLE 11
NMR Data (UHA9020)

| | UHA9020 | |
|---|---|---|
| Site | 13C | 1H |
| 1 | 139.2 | |
| 2 | 115.5 | 6.70 (1H, m) |
| 3 | 146.2 | |
| 4 | 144.4 | |
| 5 | 116.4 | 6.71 (1H, m) |
| 6 | 119.5 | 6.57 (1H, dd, J = 1.8, 7.8 Hz) |
| 7 | 43.2 | 3.43 (1H, dd-like) |
| 8 | 134.3 | 6.16 (1H, dd, J = 6.4, 16.0 Hz) |
| 9 | 129.2 | 6.26 (1H, d, J = 16.0 Hz) |
| 10 | 22.1 | 1.36 (3H, d, J = 6.9 Hz) |
| 11 | 131.5 | |
| 12 | 110.3 | 6.92 (1H, d, J = 1.9 Hz) |
| 13 | 149.0 | |
| 14 | 146.9 | |
| 15 | 116.2 | 6.72 (1H, m) |
| 16 | 120.5 | 6.77 (1H, dd, J = 1.9, 7.8 Hz) |
| 17 | 56.4 | 3.82 (3H, s) |

The physicochemical properties of the UHA9020 were as follows.

(Property)

Yellow powder

(Solubility)

### Water: Non-soluble

### Methanol: Soluble

### Ethanol: Soluble

### DMSO: Soluble

### Chloroform: Soluble

Ethyl acetate: Soluble

### Example 13

### Generation of Phenolic Polymerizable Compound from Mixture of Ferulic Acid and p-coumaric Acid

A mixed liquid (pH=7.5) obtained by dissolving 1 g of ferulic acid and 1 g of p-coumaric acid in 20 mL of ethanol and then adding 20 mL of an aqueous 5% sodium hydrogencarbonate solution was heated at 130° C. for 40 minutes in an autoclave. 1 mL of the obtained reactant was diluted with methanol in a measuring cylinder to 50 mL, and then 10 μL of the resultant reactant was analyzed by HPLC under the same conditions as those of Example 1.

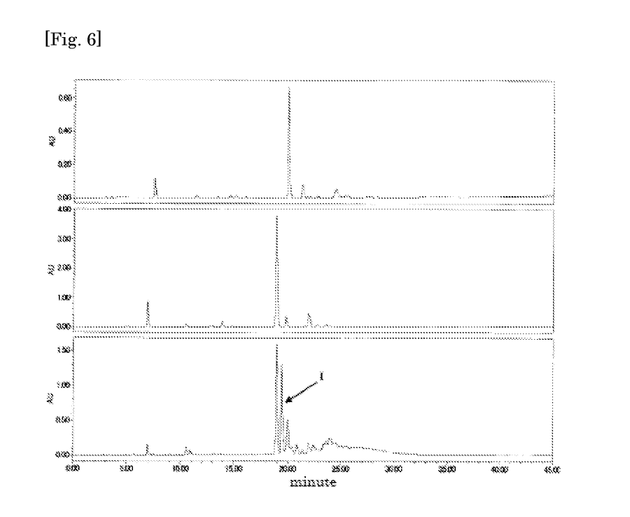

The chromatograms obtained in Example 13 are shown in FIG. 6. The upper view shows the chromatogram of the solution after the reaction of only p-coumaric acid, the middle view shows the chromatogram of the reactant of only ferulic acid, and the lower view shows the chromatogram of the solution after the mixture of p-coumaric acid and ferulic acid was generated and reacted. The peak I different from those of the UHA7009, the UHA7010, the UHA7011, and the UHA7004 was observed. Thus, the compound represented by the peak I was considered to be a product derived from both the ferulic acid and the p-coumaric acid.

### Example 14

### Isolation and Structural Determination of Phenolic Polymerizable Compound

The compounds contained in the peak shown by I of FIG. 6 obtained in the reactants obtained in Example 13 were isolated by fractionation HPLC. When the isolated HPLC eluate was dried according to a usual method, 95 mg of a yellow powdery novel compound (hereinafter referred to as UHA8015) were obtained.

Subsequently, when the molecular weight of the UHA8015 was measured by a high resolution EI-MS, the measured value was 270.3236, and the following molecular formula was obtained from the comparison with the theoretical value.

### UHA8015

Theoretical value C17H18O3 (M⁺): 270.3230
Molecular formula C₁₇H₁₈O₃

Next, the UHA8015 was subjected to NMR measurement. Then, it was confirmed that the UHA8015 had the structure represented by Formula (13) from the analysis of 1H-NMR, 13C-NMR, and various two-dimensional NMR data. This shows that the phenolic polymerizable compound represented by Formula (13) can be efficiently generated by the process of the invention.

With respect to the NMR measured values, when the sites of the UHA8015 represented by Formula (13) are as follows, the 1H nuclear magnetic resonance spectrum and the 13C nuclear magnetic resonance spectrum of each site of the UHA8015 are shown in Table 12.

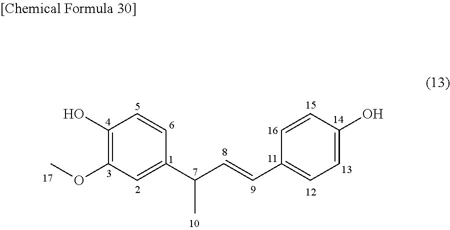 [SMILES: COc1cc(C(C)/C=C/c2ccc(O)cc2)ccc1O]

The values in the table are δ and ppm and are values measured with methanol-d3.

TABLE 12
NMR Data (UHA8015)

| | UHA8015 | |
|---|---|---|
| Site | 13C | 1H |
| 1 | 139.1 | |
| 2 | 112.1 | 6.80 (1H, d, J = 1.4 Hz) |
| 3 | 148.9 | |
| 4 | 145.7 | |
| 5 | 116.1 | 6.71 (1H, m) |
| 6 | 120.5 | 6.68 (1H, m) |
| 7 | 43.5 | 3.49 (1H, dd-like) |
| 8 | 133.8 | 6.17 (1H, dd, J = 6.9, 15.6 Hz) |
| 9 | 129.0 | 6.28 (1H, d, J = 15.6 Hz) |
| 10 | 22.1 | 1.38 (3H, d, J = 7.3 Hz) |
| 11 | 130.8 | |
| 12, 16 | 128.3 | 7.18 (2H, d, J = 8.7 Hz) |
| 13, 15 | 116.3 | 6.89 (2H, d, J = 8.7 Hz) |
| 14 | 157.7 | |
| 17 | 56.4 | 3.82 (3H, s) |

The physicochemical properties of the UHA8015 were as follows.

(Property)

Yellow powder

(Solubility)

### Water: Non-soluble

### Methanol: Soluble

### Ethanol: Soluble

### DMSO: Soluble

### Chloroform: Soluble

Ethyl acetate: Soluble

Among the phenolic polymerizable compounds obtained as described above, when the UHA7011 represented by Formula (7), the UHA6006 represented by Formula (9), the UHA9019 represented by Formula (11), the UHA9020 represented by Formula (12), and the UHA8015 represented by Formula (13) were all investigated in a known compound database (SciFinder, Japan Association for International Chemical Information), it was confirmed that the compounds are novel compounds which are not indicated in a database.

### Example 15

### Generation of Phenolic Polymerizable Compound from Mixture of Di-t-butyl Cinnamic Acid and Other 4-hydroxycinnamic Acid Compounds

(1) A mixed liquid (pH=7.8) in which 100 mg of 3,5-di-t-butyl cinnamic acid (manufactured by Wako Pure Chemical Industries, Ltd.) and 100 mg of p-coumaric acid were dissolved in 2 mL of ethanol, and 2 mL of an aqueous 5% sodium hydrogencarbonate solution was added thereto,

(2) A mixed liquid (pH=7.7) in which 100 mg of 3,5-di-t-butyl cinnamic acid and 100 mg of ferulic acid were dissolved in 2 mL of ethanol, and 2 mL of an aqueous 5% sodium hydrogencarbonate solution was added thereto,

(3) A mixed liquid (pH=7.8) in which 100 mg of 3,5-di-t-butyl cinnamic acid and 100 mg of sinapic acid were dissolved in 2 mL of ethanol, and 2 mL of an aqueous 5% sodium hydrogencarbonate solution was added thereto, and

(4) A mixed liquid (pH=5.8) in which 100 mg of 3,5-di-t-butyl cinnamic acid (manufactured by Wako Pure Chemical Industries, Ltd.) and 100 mg of coffeic acid were dissolved in 2 mL of ethanol, and 2 mL of mineral water was added thereto were individually prepared. Subsequently, each of the mixed liquids (1) to (4) was heated at 130° C. for 40 minutes in an autoclave. 1 mL of each of the obtained four kinds of reactants was diluted with methanol as appropriate, and then the resultant reactants were subjected to LC-MS or MS measurement.

The conditions of the LC-MS/MS are as follows.

Column: Negative-phase column “Develosil (Registered Trademark) C-30-UG-5” (2.0 mmi.d.×150 mm)
Mobile phase: A . . . H₂O (0.1% formic acid), B . . . Acetonitrile (0.1% formic acid)
Flow velocity: 0.2 ml/min

### Pouring: 10 μl

Detection: 3200QTRAP (Registered Trademark) LC-MS/MS system (manufactured by AB SCIEX)
Gradient (% by capacity): From 100% A/0% B to 0% A/80% B for 33 minutes and 100% B for 7 minutes (all straight line)

As a result, the molecular weight was confirmed by [M⁻] in the Negative mode. As the value of the major peak which was considered to show a phenolic polymerizable compound of each of the reactants (1) to (4),

### (1) 239 (UHA7009), 351,

### (2) 299 (UHA7004), 381,

### (3) 359 (UHA9019), 411, and

(4) 271 (UHA6006), 381 were confirmed.

The peak of the molecular weight of 463 was confirmed in the MS measurement of all of the reactants of (1) to (4).

The isolation and the structural determination for the phenolic polymerizable compounds contained in the peaks were not performed. However, from the results obtained above, it is expected that phenolic polymerizable compounds represented by the following formulae were generated in addition to the UHA7009, the UHA7004, the UHA9019, and the UHA6006.

More specifically, the compounds are shown below:

Phenolic polymerizable compound of a molecular weight 351 of (1):

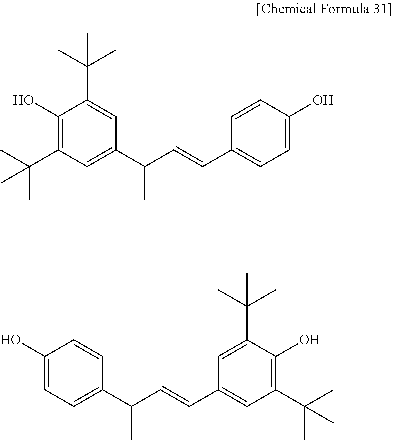 [SMILES: CC(/C=C/c1ccc(O)cc1)c1cc(C(C)(C)C)c(O)c(C(C)(C)C)c1 ; CC(/C=C/c1cc(C(C)(C)C)c(O)c(C(C)(C)C)c1)c1ccc(O)cc1]

Phenolic polymerizable compound of a molecular weight 381 of (2):

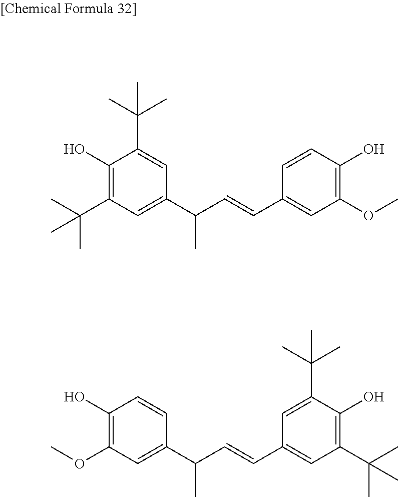

Phenolic polymerizable compound of a molecular weight 411 of (3):

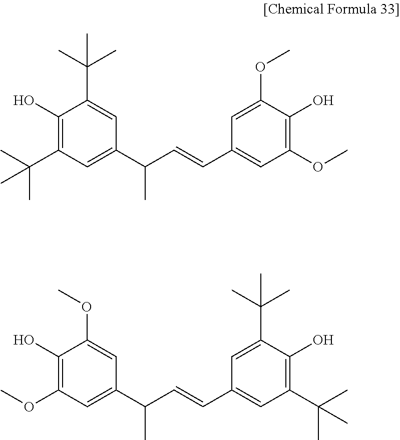

Phenolic polymerizable compound of a molecular weight 381 of (4):

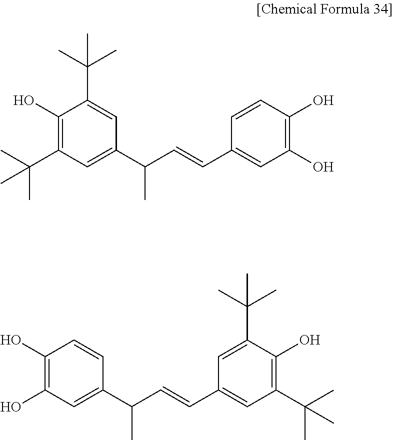 [SMILES: CC(/C=C/c1ccc(O)c(O)c1)c1cc(C(C)(C)C)c(O)c(C(C)(C)C)c1 ; CC(/C=C/c1cc(C(C)(C)C)c(O)c(C(C)(C)C)c1)c1ccc(O)c(O)c1]

Phenolic polymerizable compound of a molecular weight 463 of (1) to (4):

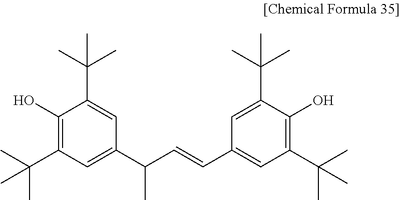 [SMILES: CC(/C=C/c1cc(C(C)(C)C)c(O)c(C(C)(C)C)c1)c1cc(C(C)(C)C)c(O)c(C(C)(C)C)c1]

### Example 16

### Generation of Phenolic Polymerizable Compound from Mixture of Artepillin C and Other 4-hydroxycinnamic Acid Compounds

(1) A mixed liquid (pH=7.9) in which 10 mg of artepillin C (manufactured by Wako Pure Chemical Industries, Ltd.) and 10 mg of p-coumaric acid were dissolved in 500 μL of ethanol, and 500 μL of an aqueous 5% sodium hydrogencarbonate solution was added thereto,

(2) A mixed liquid (pH=7.8) in which 10 mg of artepillin C and 10 mg of ferulic acid were dissolved in 500 μL of ethanol, and 500 μL of an aqueous 5% sodium hydrogencarbonate solution was added thereto,

(3) A mixed liquid (pH=7.9) in which 10 mg of artepillin C and 10 mg of sinapic acid were dissolved in 500 μL of ethanol, and 500 μL of an aqueous 5% sodium hydrogencarbonate solution was added thereto, and

(4) A mixed liquid (pH=5.7) in which 10 mg of artepillin C and 10 mg of coffeic acid were dissolved in 500 μL of ethanol, and 500 μL of mineral water was added thereto were individually prepared. Subsequently, each of the mixed liquids (1) to (4) was heated at 130° C. for 20 minutes in an autoclave. 1 mL of each of the obtained four kinds of reactants was diluted as appropriate, and then the resultant reactants were subjected to the same LC-MS or MS measurement as that of Example 15.

As a result, the molecular weight was confirmed by [M⁻] in the Negative mode. As the value of the major peak which was considered to show a phenolic polymerizable compound of each of the reactants (1) to (4),

### (1) 239 (UHA7009), 375,

### (2) 299 (UHA7004), 405,

### (3) 359 (UHA9019), 435, and

(4) 271 (UHA6006), 391 were confirmed.

The peak of the molecular weight of 511 was confirmed in the MS measurement of all of the reactants of (1) to (4).

The isolation and the structural determination for the phenolic polymerizable compounds contained in the peaks were not performed. However, from the results obtained above, it is expected that phenolic polymerizable compounds represented by the following formulae were generated in addition to the UHA7009, the UHA7004, the UHA9019, and the UHA6006.

More specifically, the compounds are shown below:

Phenolic polymerizable compound of a molecular weight of 375 of (1):

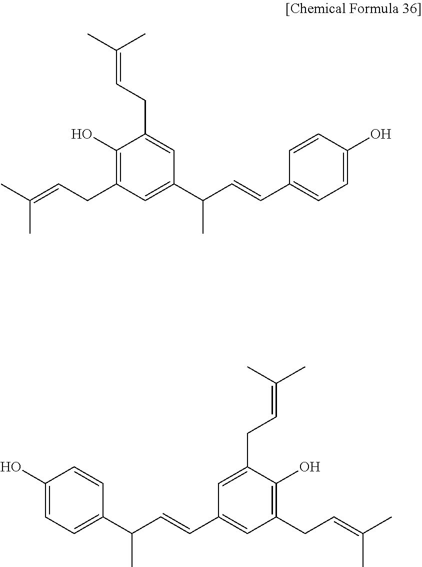 [SMILES: CC(C)=CCc1cc(C(C)/C=C/c2ccc(O)cc2)cc(CC=C(C)C)c1O ; CC(C)=CCc1cc(/C=C/C(C)c2ccc(O)cc2)cc(CC=C(C)C)c1O]

Phenolic polymerizable compound of a molecular weight of 405 of (2):

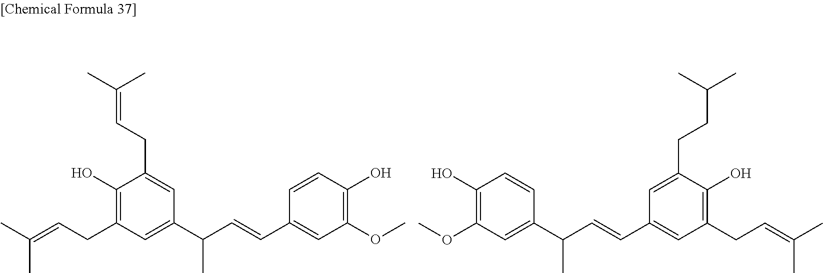

Phenolic polymerizable compound of a molecular weight of 435 of (3):

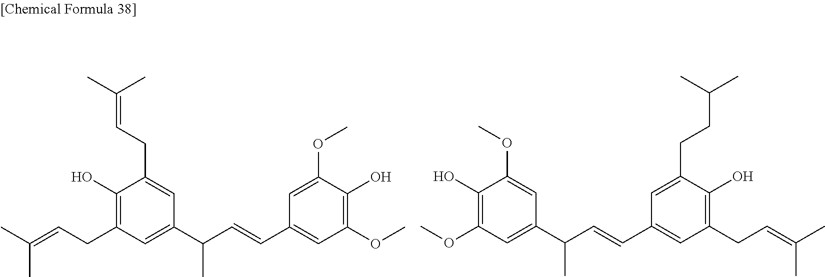

Phenolic polymerizable compound of a molecular weight of 391 of (4):

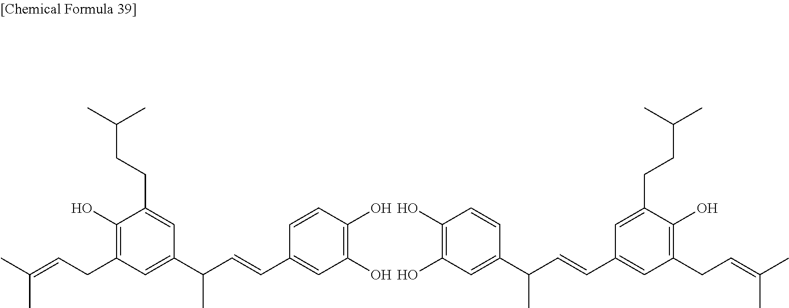

Phenolic polymerizable compounds of a molecular weight of 511 of (1) to (4):

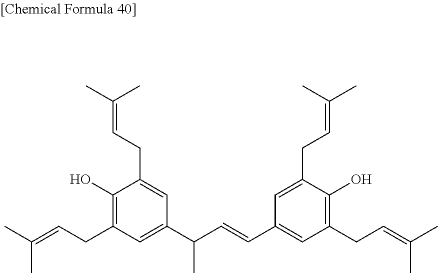 [SMILES: CC(C)=CCc1cc(/C=C/C(C)c2cc(CC=C(C)C)c(O)c(CC=C(C)C)c2)cc(CC=C(C)C)c1O]

### Example 17

### Generation of Phenolic Polymerizable Compound from Mixture of Three or More Kinds of 4-hydroxycinnamic Acid Compounds

In order to examine the reaction from three or more kinds of cinnamic acids, a mixed liquid (pH=5.3) obtained by mixing 100 mg of p-coumaric acid, 100 mg of ferulic acid, and 100 mg of coffeic acid, dissolving the mixture in 2 mL of ethanol, and then adding 2 mL of mineral water was heated at 130° C. for 20 minutes in an autoclave. 1 mL of the obtained reactant was diluted with methanol in a measuring cylinder to 50 mL. Then, 10 μL of the resultant reactant was subjected to the same LC-MS under the same conditions as those of Example 15.

As a result, even in the case of the three or more kinds of 4-hydroxycinnamic acid compounds, the molecular weight of each of the UHA6005, the UHA6006, the UHA7009, the UHA7004, the UHA7010, the UHA7011, the UHA9020, and the UHA8015 was confirmed and it was also confirmed that the phenolic polymerizable compound was generated even from the mixture of the three or more kinds of 4-hydroxycinnamic acid compounds.

From the results of Example 17, it was confirmed that, even in the case of the reaction of the mixed liquid of various kinds of compounds, the reaction proceeded. This shows that the application and the use thereof to a random library can be expected.

### Example 18

### Anti-Cancer Action of Phenolic Polymerizable Compound

Next, in order to see the effect of the phenolic polymerizable compound to cancer cells, cancer cell growth suppressing action using HL-60 cells (human promyelocytic leukemia cells) was tested.

For culturing the HL-60 cells, an enriched culture medium “RPMI-1640” (manufactured by Sigma-Aldrich Japan) containing 4 mM glutamine (L-Glutamine, manufactured by Sigma-Aldrich Japan) and 10% FBS (Foetal Bovine Serum, manufactured by Biological Industries) was used. For the test, a 96-well plate for cell culture (manufactured by Corning Japan) was used, and the HL-60 cells whose number of cells was adjusted to be 5×10⁵ cells/mL were seeded at 100 μL per well.

As samples, p-coumaric acid, ferulic acid, coffeic acid, and sinapic acid, and the UHA6005, the UHA6006, the UHA7004, the UHA7009, the UHA7010, the UHA7011, the UHA8015, the UHA9019, and the UHA9020 which were already purified were used. With respect to the preparation of the samples, each compound was dissolved in DMSO (Wako Pure Chemical Industries, Ltd.), and then adjusted in such a manner that the final concentration in the HL-60 cell culture solutions was 6.3 μM, 12.5 μM, 25 μM, 50 μM, and 100 μM. Then, the test was started.

The number of viable cells was quantified by an MTT method using a “Cell counting kit-8” (manufactured by DOJINDO LABORATORIES). 24 hours after starting the test, 10 μl of a Cell counting kit-8 solution was added to each well, and then sufficiently stirred. After a shading reaction for 1 hour, the absorbancy was measured at a measurement wavelength of 450 nm using a plate leader (Biorad Laboratories, Inc., “BIO-RAD Model 680”). Then, the cell viability was calculated based on the obtained data. The cell viability is a value obtained by setting the number of viable cells of the culture liquid to which only DMSO as a solvent was added to 100%, and then calculating the number of viable cells under the concentration of each compound as a relative value. From the relationship between the concentration of each compound and the cell viability, the concentration IC₅₀ (50% inhibition concentration) at which the cell growth is suppressed by 50% was calculated. The results are shown in Table 13. From these results, a cancer cell growth suppressing ability higher than that of the 4-hydroxycinnamic acid compounds and hydroxystilbenes which are the raw materials was observed in each of the phenolic polymerizable compounds.

TABLE 13

| | Cell growth suppressing ability |
|---|---|
| | to HL-60 (IC50, μM) |
| p-coumaric acid | >100 |
| UHA7009 | 49.3 |
| UHA7010 | 35.3 |
| UHA7011 | 35.4 |
| Coffeic acid | >100 |
| UHA6005 | 43.7 |
| UHA6006 | 42.4 |
| Ferulic acid | >100 |
| UHA7004 | 17.6 |
| Sinapic acid | >100 |
| UHA9019 | 66.2 |
| UHA8015 | 74.3 |
| UHA9020 | 38.5 |

### Example 19

### Anti-Cancer Action to Oral Cancer of Phenolic Polymerizable Compound

Next, in order to see the effect of the phenolic polymerizable compound to oral cancer cells, cancer cell growth suppressing action using SCC-4 cells (human oral cancer cells, ATCC) was tested.

For culturing the SCC-4 cells, a DMEM/F-12 (1:1) culture medium (manufactured by GIBCO) containing 400 ng/mL hydrocortisone (manufactured by Sigma-Aldrich Japan), 1% antibiotic-antimycotic (manufactured by GIBCO), and 10% FBS (Foetal Bovine Serum, manufactured by ATCC) was used. For the test, a collagen I coat 96 well plate for cell culture (manufactured by Japan BD) was used, and SCC-4 cells whose number of cells was adjusted to be 5×10⁵ cells/mL were seeded at 100 μL per well. The cells were cultured under the conditions of 37° C. and 5% CO₂ for 24 hours, and were used for the test in a confluent state of 80% or higher.

As samples, p-coumaric acid and coffeic acid and the UHA6005, the UHA7009, the UHA7010, and the UHA7011 which were already purified were used. With respect to the preparation of the samples, each compound was dissolved in DMSO, and then prepared in such a manner as to achieve 0.63 mM, 1.25 mM, 2.5 mM, 5 mM, and 10 mM. The samples were added in such a manner that the final concentration in the SCC-4 cell culture solution was 6.3 μM, 12.5 μM, 25 μM, 50 μM, and 100 μM. Then, the test was started. A sample to which an equivalent amount of only DMSO as the solvent was added was used as a negative control.

The number of viable cells was quantified by an MTT method using a “Cell counting kit-8” in the same manner as in Example 18, and the concentration IC₅₀ at which the cell growth is suppressed by 50% was calculated. The results are shown in Table 14. From these results, a cancer cell growth suppressing ability higher than that of the 4-hydroxycinnamic acid compounds and hydroxystilbenes which are the raw materials was observed in each of the phenolic polymerizable compounds.

TABLE 14

| | Cell growth suppressing |
|---|---|
| Compound | ability (IC₅₀, μM) |
| p-coumaric acid | >100 |
| UHA7009 | 72.9 |
| UHA7010 | 61.1 |
| UHA7011 | 40.8 |
| Coffeic acid | >100 |
| UHA6005 | 69.7 |

### Example 20

### Lipase Inhibitory Action of Phenolic Polymerizable Compound

In order to see the inhibitory action to lipase of the phenolic polymerizable compound, an inhibitory action test using rat intestine derived lipase was performed.

As lipase, one obtained by suspending 100 mg of rat derived intestine acetone powder (manufactured by Sigma-Aldrich Japan) in 1 ml of 100 mM citric acid buffer (pH 6.0), stirred at 4° C. for 1 hour, centrifuging the same (at 15000 rpm for 45 minutes at 4° C.), and then diluting the supernatant by 1500 times was used as a lipase solution.

As samples, p-coumaric acid, ferulic acid, and coffeic acid and the UHA6005, the UHA6006, the UHA7009, the UHA7010, the UHA7011, the UHA8015, and the UHA9020 which were already purified were used. With respect to the preparation of the samples, each compound was dissolved in DMSO, and then prepared in such a manner as to achieve 0.1 mM, 0.5 mM, 1 mM, 2 mM, and 4 mM.

For measuring the activity, a “Lipase kit S” (Trade name, manufactured by Dainippon Pharmaceutical Co., Ltd.) was used. First, in accordance with a preparation method described in the catalog of the Lipase kit S, a color developing liquid was prepared. Reaction liquids in which 70 μl of the color developing liquid, 2 μl of an esterase inhibitor, 10 μl of the lipase solution, and 10 μl of the samples (Final concentration: 10 μM, 50 μM, 100 μM, 200 μM, 400 μM, and 1000 μM) were mixed were prepared, preincubated at 30° C. for 5 minutes, and then 8 μl of a substrate solution described in the catalog was added. Then, a reaction was started. After reacting for 10 minutes, 150 μl of a reaction stop liquid prepared in accordance with the preparation method described in the catalog of the Lipase kit S was added to stop the reaction. The resultant substance was subjected to a measurement of absorbancy at a wavelength of 415 nm. A reaction liquid to which only DMSO as the solvent of the sample was added was used as a positive control and one to which 10 μl of 100 mM citric acid buffer (pH 6.0) was added instead of the lipase solution was used as a negative control. From the relationship between the lipase inhibition rate calculated based on the data obtained therefrom and the concentration of each compound, the concentration IC₅₀ at which the lipase activity is inhibited by 50% was calculated. The results are shown in Table 15.

TABLE 15

| | Inhibitory action to |
|---|---|
| | lipase (IC50, μM) |
| p-coumaric acid | >1000 |
| UHA7009 | 438.0 |
| UHA7010 | 60.4 |
| UHA7011 | 197.1 |
| Coffeic acid | >1000 |
| UHA6005 | 253.1 |
| UHA6006 | 187.7 |
| Ferulic acid | >1000 |
| UHA8015 | 350.0 |
| UHA9020 | 118.5 |

From these results, a lipase inhibition activity higher than that of the raw materials was observed in the phenolic polymerizable compounds. Accordingly, since the phenolic polymerizable compounds demonstrate outstanding lipase inhibitory action, it is considered that the compounds are useful as anti-obesity agents and also as metabolic syndrome prevention agents. Since the lipase inhibition on the skin is effective for prevention of pimples and recovery from pimples, it is considered that the compounds are useful also as skin disease therapeutic agents for prevention of pimples, recovery of pimples, and the like.

Hereinafter, compounding examples of the phenolic polymerizable compounds represented by Formulae (5), (6), and (7) obtained using p-coumaric acid as the raw materials are described as Examples of extracts containing the phenolic polymerizable compound, foods containing the extracts containing the phenolic polymerizable compound, pharmaceutical agents containing the phenolic polymerizable compound, quasi-drugs containing phenolic polymerizable compound, and cosmetics containing the phenolic polymerizable compound but it is a matter of course that other phenolic polymerizable compounds can be similarly used.

As substances containing the 4-hydroxycinnamic acid compound serving as the raw materials, propolis extracts and the like may be used as p-coumaric acid and artepillin C, coffee and SHIMON tea (dried product of sweet potato leaf portion) and enzyme-treated substances thereof may be used as coffeic acid, ferulic acid and rice bran extracts of food additives may be used as ferulic acid, and extracts of mustard, Japanese horseradish, and the like, enzyme-treated substances thereof, and the like, may be used as sinapic acid but the substances containing the 4-hydroxycinnamic acid compound are not limited thereto.

### Example 21

### Preparation of Extracts Containing UHA7009, UHA7010, and UHA7011

A mixed solution (pH=8.5) prepared by adding 10 g of a propolis water-extracted extract, 10 ml of ethanol, and 1 g of sodium bicarbonate was heated at 130° C. for 60 minutes in an autoclave. The obtained reaction solution was heated under reduced pressure to dry the same for solidification, thereby obtaining 11 g of an extract containing UHA7009, UHA7010, and UHA7011. In the obtained extract containing UHA7009, UHA7010, and UHA7011, 0.015 g of the UHA7009, 0.001 g of the UHA7010, and 0.003 g of the UHA7011 were contained as confirmed by the same technique as that of Example 2. The generation operation was repeated as required, thereby further obtaining the extracts.

### Example 22

### Food Containing UHA7009, UHA7010, and UHA7011

1 g of the extract containing UHA7009, UHA7010, and UHA7011 obtained in Example 21 was dissolved in 100 mL of ethanol beforehand, 500 g of sugar and 400 g of starch syrup were mixed and dissolved in the solution, and 100 g of fresh cream, 20 g of butter, 70 g of condensed milk, and 1.0 g of emulsifier were mixed in the mixture. Then, the pressure was reduced to −550 mmHg in a vacuum pan, and then the resultant mixture was condensed under the conditions of 115° C., thereby obtaining a milk hard candy having a moisture value of 3.0% by weight. It is a matter of course that the milk hard candy is easy to eat as a confectionery. Moreover, since the UHA7009, the UHA7010, and the UHA7011 are contained, the milk hard candy can be used also as a functional food which is expected to improve obesity, to prevent obesity, to reduce the risk of diffusion of cancer in cancer patients, to reduce the risk of the onset of cancer, and to prevent cancer.

### Example 23

### Pharmaceutical Agent Containing UHA7009

The powder of the UHA7009 obtained by the same process as that of Examples 1 and 2 was dissolved in ethanol, adsorbed to microcrystalline cellulose, and then dried under reduced pressure. The resultant substance was treated according to a usual method, thereby obtaining a tablet product. The formula is as follows: 10 parts by weight of UHA7009 powder, 23 parts by weight of cornstarch, 12 parts by weight of milk sugar, 8 parts by weight of carboxymethyl cellulose, 32 parts by weight of microcrystalline cellulose, 4 parts by weight of polyvinyl pyrrolidone, 3 parts by weight of magnesium stearate, and 8 parts by weight of talc. Since the tablet product contains the UHA7009, the tablet product can be effectively used as a pharmaceutical agent aiming at improvement of obesity, prevention of obesity, and recovery from cancer.

### Example 24

### Quasi-Drug Containing UHA7009

1.2 g of the powder of UHA7009 obtained by the process of Examples 1 and 2 was dissolved in 10 mL of ethanol, one in which 20 g of taurine, 0.12 g of vitamin B1 nitrate, 0.6 g of sodium benzoate, 4 g of citric acid, and 10 g of polyvinyl pyrrolidone were all dissolved in purified water was mixed in the same, and then the resultant substance was diluted with purified water in a measuring cylinder to 1000 mL. The pH of the obtained solution was adjusted to 3.2 using dilute hydrochloric acid. 50 mL of 1000 mL of the obtained solution was charged into a glass bottle, and then sterilized at 80° C. for 30 minutes, thereby completing a drink agent which is a quasi-drug. Since the drink agent contains the UHA7009, the drink agent can be effectively used as a quasi-drug which is expected to improve obesity, to prevent obesity, to reduce the risk of diffusion of cancer in cancer patients, to reduce the risk of the onset of cancer, and to prevent cancer in addition to the purpose of supply of nutrients.

### Example 25

### Cosmetics Containing UHA7010

1 part by weight of tetraoleic acid polyoxyethylene sorbitol, 0.5 part by weight of polyoxyethylene stearyl ether, 1 part by weight of lipophilic glyceryl monostearate, 0.5 part by weight of pyruvic acid, 0.5 part by weight of stearyl alcohol, 1 part by weight of avocado oil, and 0.1 part by weight of powder of UHA7010 obtained by the same process as that of Examples 1 and 2 were mixed, the mixture was dissolved according to a usual method, 1 part by weight of sodium lactate, 5 parts by weight of propylene glycol, 0.1 part by weight of carboxy vinyl polymer, a very slight amount of spice, and 89.3 parts by weight of purified water were added thereto, and the mixture was treated by a homogenizer for emulsification, thereby obtaining a milky lotion. Since the milky lotion contains the UHA7010, the milky lotion can be effectively used as medicated cosmetics having medical treatment and prevention effects of skin diseases, such as pimples.

1. A process for producing a phenolic polymerizable compound represented by Formula (1): 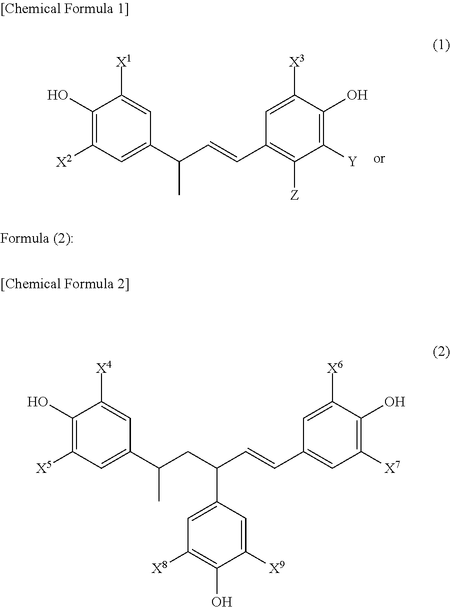
wherein X¹-X⁹ represent a hydrogen atom, a hydroxy group, a saturated or unsaturated linear or branched alkoxy group having 1-10 carbon atoms, or a saturated or unsaturated linear or branched alkyl group having 1-10 carbon atoms;
Y represents a hydrogen atom, a hydroxy group, a saturated or unsaturated linear or branched alkoxy group having 1-10 carbon atoms, a saturated or unsaturated linear or branched alkyl group having 1-10 carbon atoms, or a group represented by Formula (3): 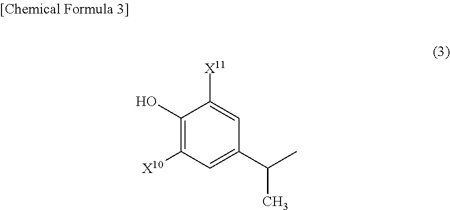
wherein X¹⁰ and X¹¹ represent a hydrogen atom, a hydroxy group, a saturated or unsaturated linear or branched alkoxy group having 1-10 carbon atoms, or a saturated or unsaturated linear or branched alkyl group having 1-10 carbon atoms; and
Z represents a hydrogen atom or a group represented by Formula (3);
wherein X¹-X¹¹ may be the same or different from one another),
the process which is characterized by heating 4-hydroxycinnamic acid compounds in the presence of a metal salt.

2. The process according to claim 1, wherein the 4-hydroxycinnamic acid compounds are represented by the following formula (4): 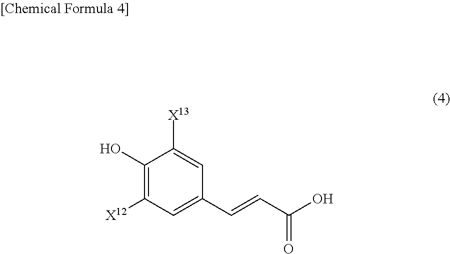
wherein X¹² and X¹³ represent a hydrogen atom, a hydroxy group, a saturated or unsaturated linear or branched alkoxy group having 1-10 carbon atoms, or a saturated or unsaturated linear or branched alkyl group having 1-10 carbon atoms, and X¹² and X¹³ may be the same or different from each other.

3. The process according to claim 2, wherein the 4-hydroxycinnamic acid compounds are one or more kinds of compounds selected from a group consisting of p-coumaric acid, ferulic acid, coffeic acid, sinapic acid, di-t-butyl hydroxycinnamic acid compound, and artepillin C.

4. The process according to claim 3, wherein the phenolic polymerizable compound generated by heating p-coumaric acid in the presence of a metal salt is a compound represented by Formula (5):

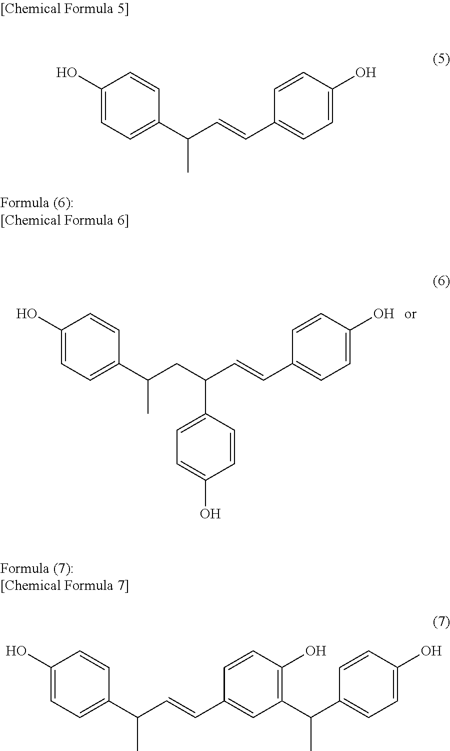 [SMILES: CC(/C=C/c1ccc(O)cc1)c1ccc(O)cc1 ; CC(CC(/C=C/c1ccc(O)cc1)c1ccc(O)cc1)c1ccc(O)cc1 ; CC(/C=C/c1ccc(O)c(C(C)c2ccc(O)cc2)c1)c1ccc(O)cc1]

5. The process according to claim 3, wherein a phenolic polymerizable compound generated by heating coffeic acid in the presence of a metal salt is a compound represented by Formula (8):

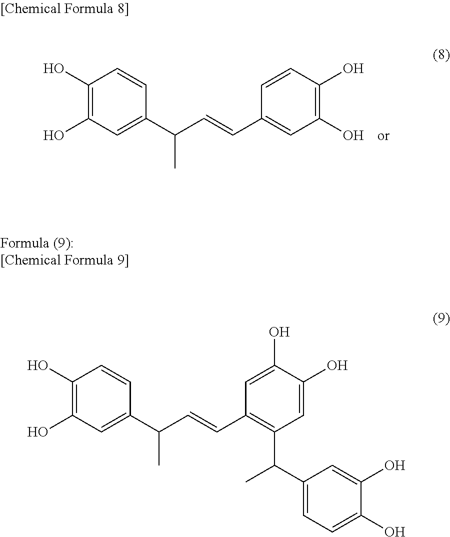 [SMILES: CC(/C=C/c1ccc(O)c(O)c1)c1ccc(O)c(O)c1 ; CC(/C=C/c1cc(O)c(O)cc1C(C)c1ccc(O)c(O)c1)c1ccc(O)c(O)c1]

6. The process according to claim 3, wherein the phenolic polymerizable compound generated by heating ferulic acid in the presence of a metal salt is a compound represented by Formula (10):

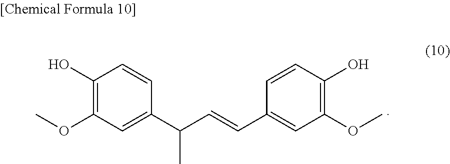

7. The process according to claim 3, wherein the phenolic polymerizable compound generated by heating sinapic acid in the presence of a metal salt is a compound represented by Formula (11):

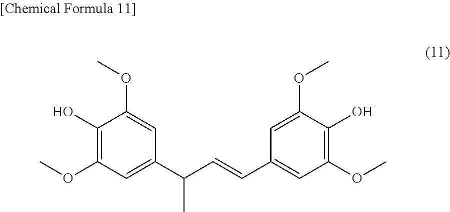

8. The process according to claim 3, wherein a phenolic polymerizable compound generated by heating coffeic acid and ferulic acid in the presence of a metal salt is a compound represented by Formula (12):

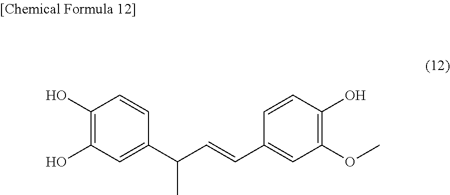 [SMILES: COc1cc(/C=C/C(C)c2ccc(O)c(O)c2)ccc1O]

9. The process according to claim 3, wherein the phenolic polymerizable compound generated by heating p-coumaric acid and ferulic acid in the presence of a metal salt is a compound represented by Formula (13):

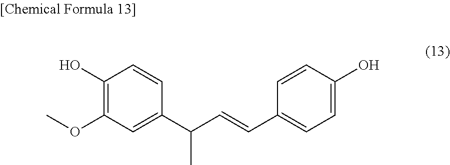 [SMILES: COc1cc(C(C)/C=C/c2ccc(O)cc2)ccc1O]

10. The process according to claim 1, comprising performing heat treatment at 90° C. to 150° C.

11. An anticancer agent, comprising a phenolic polymerizable compound produced by the process according to claim 1.

12. An anticancer agent to oral cancer, comprising a phenolic polymerizable compound produced by the process according to claim 1.

13. A lipase inhibitor, comprising a phenolic polymerizable compound produced by the process according to claim 1.

14. An anti-obesity agent, comprising a phenolic polymerizable compound produced by the process according to claim 1.

15. A skin disease therapeutic agent, comprising a phenolic polymerizable compound produced by the process according to claim 1.

16. A food, a pharmaceutical agent, a quasi-drug, or cosmetics, comprising a phenolic polymerizable compound produced by the process according to claim 1.

17. A novel physiologically active phenolic polymerizable compound represented by Formula (7) or a pharmacologically permissible salt thereof:

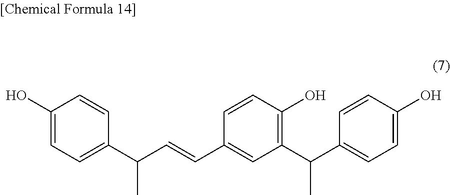 [SMILES: CC(/C=C/c1ccc(O)c(C(C)c2ccc(O)cc2)c1)c1ccc(O)cc1]

18. A novel physiologically active phenolic polymerizable compound represented by Formula (9) or a pharmacologically permissible salt thereof:

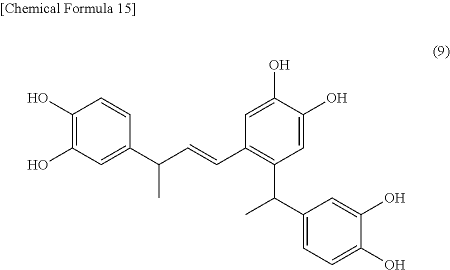 [SMILES: CC(/C=C/c1cc(O)c(O)cc1C(C)c1ccc(O)c(O)c1)c1ccc(O)c(O)c1]

19. A novel physiologically active phenolic polymerizable compound represented by Formula (11) or a pharmacologically permissible salt thereof:

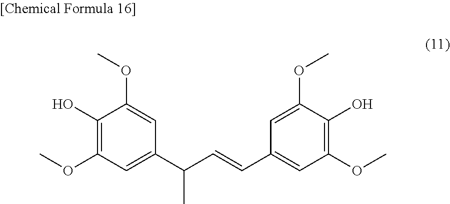

20. A novel physiologically active phenolic polymerizable compound represented by Formula (12) or a pharmacologically permissible salt thereof:

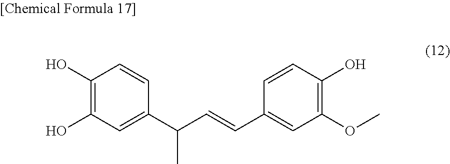 [SMILES: COc1cc(/C=C/C(C)c2ccc(O)c(O)c2)ccc1O]

21. A novel physiologically active phenolic polymerizable compound represented by Formula (13) or a pharmacologically permissible salt thereof: 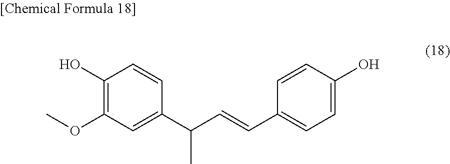 [SMILES: COc1cc(C(C)/C=C/c2ccc(O)cc2)ccc1O]
